(19) United States
(12) Patent Application Publication
(10) Pub. No.: US 2022/0013195 A1
(43) Pub. Date: Jan. 13, 2022

(54) SYSTEMS AND METHODS FOR ACCESS MANAGEMENT AND CLUSTERING OF GENOMIC OR PHENOTYPE DATA

(71) Applicant: IX LAYER INC., Atherton, CA (US)

(72) Inventors: Pouria SANAE, Atherton, CA (US); Vahid KOWSARI, Atherton, CA (US)

(21) Appl. No.: 17/380,563

(22) Filed: Jul. 20, 2021

Related U.S. Application Data
(63) Continuation of application No. PCT/US2020/014471, filed on Jan. 21, 2020.
(60) Provisional application No. 62/795,283, filed on Jan. 22, 2019.

(51) Int. Cl. G16B 50/30; G16B 45/00; G16B 20/00; G06F 21/31

(52) U.S. Cl. CPC G16B 50/30; G16B 45/00; G06F 2221/2141; G06F 21/31; G16B 20/00

(57) ABSTRACT

The present disclosure provides systems and methods for facilitating secure and convenient genetic data exchange among different users. A computer-implemented method for cloud-based genomic or phenotype data access among a plurality of digital computers may comprise a first digital computer of a first user and a second digital computer of a second user, comprising: (a) providing a cloud-based computer system comprising a network interface that is in network communication with the first and second digital computers; (b) through the network interface, receiving a request from the first digital computer to provide the second user access to a set of genomic or phenotype data, which set of genomic or phenotype data is generated from processing biological samples of a subject; and (c) subsequent to receiving said request in (b), permitting the second user to access at least a subset of the set of genomic or phenotype data through the second computer.

### CROSS-REFERENCE

This application is a continuation of International Application No. PCT/US2020/014471, filed Jan. 21, 2020, which claims the benefit of U.S. Provisional Patent Application No. 62/795,283, filed Jan. 22, 2019, each of which is entirely incorporated by reference herein.

### BACKGROUND

A large number of disorders or diseases may each have their own unique characteristic genetic basis. Thus, analysis of genetic or phenotype data of human subjects may provide valuable insights into disease cause and risk as well as drug discovery and development in various physiology-related fields. However, launching genetic products can be expensive, complicated, and time-consuming.

### SUMMARY

The present disclosure is related to access management (e.g., sharing among multiple users and/or entities) and clustering of genomic or phenotype data. Although analysis of human genetic data may significantly advance our understanding of diseases, there can be concerns about genetic data sharing or disclosure of human subjects. In addition, there may be incomplete oversight of genetic testing or data analysis.

The present disclosure provides systems and methods which may advantageously enable secure, efficient, and convenient access management (e.g., sharing among multiple users and/or entities) and clustering of human genomic or phenotype data. The systems and methods of the present disclosure can be cloud-based. Such secure, efficient, and convenient access management and clustering of human genomic or phenotype data can advantageously accelerate scientific discovery with high cost efficiencies. For example, healthcare, wellness, and nutrition entities can leverage systems and methods of the present disclosure to provide direct-to-consumer genetic products that add the value of personalization based on users' DNA. The systems and methods of the present disclosure may greatly facilitate removal of barriers such as technology and regulatory, thereby enabling different entities to launch genetic products to end-consumers in a user-friendly way.

In an aspect, the present disclosure provides a computer-implemented method for cloud-based genomic or phenotype data access among a plurality of digital computers comprising a first digital computer of a first user and a second digital computer of a second user, comprising: (a) providing a cloud-based computer system comprising a network interface that is in network communication with the first digital computer of the first user and the second digital computer of the second user; (b) through the network interface, receiving a request from the first digital computer to provide the second user access to a set of genomic or phenotype data, which set of genomic or phenotype data is generated from processing at least one biological sample of a subject; and (c) subsequent to receiving the request in (b), permitting the second user to access at least a subset of the set of genomic or phenotype data through the second computer of the second user.

In some embodiments, the genomic data may include genetic data such as DNA sequence information, RNA sequence information, and/or protein sequence information. In some embodiments, the phenotype data comprises Electronic Health Record (EHR) data of one or more subjects (e.g., patients). In some embodiments, operation (c) comprises transferring the at least the subset of the set of genomic or phenotype data to the second computer. In some embodiments, the set of genomic or phenotype data can be stored in the cloud-based computer system, and operation (c) comprises (i) permitting the second user to access the at least the subset of the set of genomic or phenotype data in the cloud-based computer system or (ii) transferring the at least the subset of the set of genomic or phenotype data from the cloud-based computer system to the second computer. In some embodiments, the method further comprises, prior to operation (c), receiving at the cloud-based computer system the set of genomic or phenotype data from the first digital computer. In some embodiments, the method further comprises receiving at the cloud-based computer system a second set of genomic or phenotype data from the second digital computer, which second set of genomic or phenotype data is generated from at least one biological sample of the subject. In some embodiments, the second set of genomic or phenotype data is different than the first set of genomic or phenotype data. In some embodiments, the first user is the subject. In some embodiments, the second user is the subject. The method herein may further comprise receiving an item of value from the second user in exchange for permitting the second user to access the at least the subset of the set of genomic or phenotype data. In some embodiments, the method further comprises providing at least a portion of the item of value to the first user. In some embodiments, the first user may be associated with a first company and the second user may be associated with a second company different from the first company. In some embodiments, the first user may be the subject and the second user may be associated with a company. In some embodiments, operation (b) further comprises using an account of the first user. In some embodiments, the at least the subset of the set of genomic or phenotype data is configured to be used by the second user or a third user to generate health-related information of the subject. In some embodiments, the method further comprises communicating the health-related information of the subject to the first user. In some embodiments, the first user may be the subject or the second user may be the subject. In some embodiments, the method further comprises allowing the first user to manage the set of genomic or phenotype data through the network interface, wherein managing the set of genomic or phenotype data comprises granting access to one or more additional users, reviewing access by the one or more additional users, or manipulating the set of genomic or phenotype data. In some embodiments, the network interface comprises a graphical user interface (GUI). In some embodiments, the network interface is provided via a mobile or web application. In some embodiments, the set of genomic or phenotype data is stored on a private cloud of the first user. In some embodiments, the private cloud comprises a private database structure.

In another aspect, the present disclosure provides a cloud-based method for facilitating genomic or phenotype data exchange, comprising permitting a first entity to access genomic or phenotype data of a second entity over a cloud-based computer system, wherein the genomic or phenotype data is generated from processing at least one biological sample of a subject. In some embodiments, the permission is provided by the second entity. In some embodiments, the permission is provided by the cloud-based computer system. In some embodiments, the cloud-based computer system comprises a network interface. In some embodiments, the set of genomic or phenotype data is configured to be used by the second entity or a third entity to generate health-related information of the subject.

In another aspect, the present disclosure provides a computer system for cloud-based genomic or phenotype data access among a plurality of digital computers comprising a first digital computer of a first user and a second digital computer of a second user, comprising: a cloud-based computer system comprising a network interface that is in network communication with said first digital computer of said first user and said second digital computer of said second user; and one or more computer processors operatively coupled to said cloud-based computer system, wherein said one or more computer processors are individually collectively programmed to: (i) through said network interface, receive a request from said first digital computer to provide said second user access to a set of genomic or phenotype data, which set of genomic or phenotype data is generated from processing at least one biological sample of a subject; and (ii) subsequent to receiving said request, permit said second user to access at least a subset of said set of genomic or phenotype data through said second computer of said second user.

In some embodiments, the genomic data may include genetic data such as DNA sequence information, RNA sequence information, and/or protein sequence information. In some embodiments, the phenotype data comprises Electronic Health Record (EHR) data of one or more subjects (e.g., patients). In some embodiments, operation (ii) comprises transferring the at least the subset of the set of genomic or phenotype data to the second computer. In some embodiments, the set of genomic or phenotype data can be stored in the cloud-based computer system, and operation (ii) comprises (1) permitting the second user to access the at least the subset of the set of genomic or phenotype data in the cloud-based computer system or (2) transferring the at least the subset of the set of genomic or phenotype data from the cloud-based computer system to the second computer. In some embodiments, the one or more computer processors are individually collectively programmed to further, prior to operation (ii), receive at the cloud-based computer system the set of genomic or phenotype data from the first digital computer. In some embodiments, the one or more computer processors are individually collectively programmed to further receive at the cloud-based computer system a second set of genomic or phenotype data from the second digital computer, which second set of genomic or phenotype data is generated from at least one biological sample of the subject. In some embodiments, the second set of genomic or phenotype data is different than the first set of genomic or phenotype data. In some embodiments, the first user is the subject. In some embodiments, the second user is the subject. The one or more computer processors may be individually collectively programmed to further receive an item of value from the second user in exchange for permitting the second user to access the at least the subset of the set of genomic or phenotype data. In some embodiments, the one or more computer processors are individually collectively programmed to further provide at least a portion of the item of value to the first user. In some embodiments, the first user may be associated with a first company and the second user may be associated with a second company different from the first company. In some embodiments, the first user may be the subject and the second user may be associated with a company. In some embodiments, operation (i) further comprises using an account of the first user. In some embodiments, the at least the subset of the set of genomic or phenotype data is configured to be used by the second user or a third user to generate health-related information of the subject. In some embodiments, the one or more computer processors are individually collectively programmed to further communicate the health-related information of the subject to the first user. In some embodiments, the first user may be the subject or the second user may be the subject. In some embodiments, the one or more computer processors are individually collectively programmed to further allow the first user to manage the set of genomic or phenotype data through the network interface, wherein managing the set of genomic or phenotype data comprises granting access to one or more additional users, reviewing access by the one or more additional users, or manipulating the set of genomic or phenotype data. In some embodiments, the network interface comprises a graphical user interface (GUI). In some embodiments, the network interface is provided via a mobile or web application. In some embodiments, the set of genomic or phenotype data is stored on a private cloud of the first user. In some embodiments, the private cloud comprises a private database structure.

In another aspect, the present disclosure provides a computer system for facilitating genomic or phenotype data exchange, comprising one or more computer processors operatively coupled to a cloud-based computer system, wherein the one or more computer processors are individually collectively programmed to permit a first entity to access genomic or phenotype data of a second entity over a cloud-based computer system, wherein the genomic or phenotype data is generated from processing at least one biological sample of a subject. In some embodiments, the permission is provided by the second entity. In some embodiments, the permission is provided by the cloud-based computer system. In some embodiments, the cloud-based computer system comprises a network interface. In some embodiments, the set of genomic or phenotype data is configured to be used by the second entity or a third entity to generate health-related information of the subject.

In an aspect, the present disclosure provides a non-transitory computer-readable medium comprising machine-executable code that, upon execution by one or more computer processors, implements a method for cloud-based genomic or phenotype data access among a plurality of digital computers comprising a first digital computer of a first user and a second digital computer of a second user, the method comprising: (a) providing a cloud-based computer system comprising a network interface that is in network communication with the first digital computer of the first user and the second digital computer of the second user; (b) through the network interface, receiving a request from the first digital computer to provide the second user access to a set of genomic or phenotype data, which set of genomic or phenotype data is generated from processing at least one biological sample of a subject; and (c) subsequent to receiving the request in (b), permitting the second user to access at least a subset of the set of genomic or phenotype data through the second computer of the second user.

In some embodiments, the genomic data may include genetic data such as DNA sequence information, RNA sequence information, and/or protein sequence information. In some embodiments, the phenotype data comprises Electronic Health Record (EHR) data of one or more subjects (e.g., patients). In some embodiments, operation (c) comprises transferring the at least the subset of the set of genomic or phenotype data to the second computer. In some embodiments, the set of genomic or phenotype data can be stored in the cloud-based computer system, and operation (c) comprises (i) permitting the second user to access the at least the subset of the set of genomic or phenotype data in the cloud-based computer system or (ii) transferring the at least the subset of the set of genomic or phenotype data from the cloud-based computer system to the second computer. In some embodiments, the method further comprises, prior to operation (c), receiving at the cloud-based computer system the set of genomic or phenotype data from the first digital computer. In some embodiments, the method further comprises receiving at the cloud-based computer system a second set of genomic or phenotype data from the second digital computer, which second set of genomic or phenotype data is generated from at least one biological sample of the subject. In some embodiments, the second set of genomic or phenotype data is different than the first set of genomic or phenotype data. In some embodiments, the first user is the subject. In some embodiments, the second user is the subject. The method herein may further comprise receiving an item of value from the second user in exchange for permitting the second user to access the at least the subset of the set of genomic or phenotype data. In some embodiments, the method further comprises providing at least a portion of the item of value to the first user. In some embodiments, the first user may be associated with a first company and the second user may be associated with a second company different from the first company. In some embodiments, the first user may be the subject and the second user may be associated with a company. In some embodiments, operation (b) further comprises using an account of the first user. In some embodiments, the at least the subset of the set of genomic or phenotype data is configured to be used by the second user or a third user to generate health-related information of the subject. In some embodiments, the method further comprises communicating the health-related information of the subject to the first user. In some embodiments, the first user may be the subject or the second user may be the subject. In some embodiments, the method further comprises allowing the first user to manage the set of genomic or phenotype data through the network interface, wherein managing the set of genomic or phenotype data comprises granting access to one or more additional users, reviewing access by the one or more additional users, or manipulating the set of genomic or phenotype data. In some embodiments, the network interface comprises a graphical user interface (GUI). In some embodiments, the network interface is provided via a mobile or web application. In some embodiments, the set of genomic or phenotype data is stored on a private cloud of the first user. In some embodiments, the private cloud comprises a private database structure.

Another aspect of the present disclosure provides a non-transitory computer-readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A shows an example of a client virtual private cloud (VPC), which can be implemented using a system for facilitating genomic or phenotype data exchange.

FIGS. 1B and 1C show examples of how a core platform can interface with each of a plurality of VPCs.

FIG. 1D shows an example of the core platform has multiple functionalities integrated with each client VPC.

FIG. 1E shows an example of a system for facilitating genomic or phenotype data exchange, in this case, a system that allows genetic data exchange between two different companies.

FIG. 1F shows an example of a system for facilitating genomic or phenotype data exchange, in this case, a system that allows genetic data upload by the user that can be selectively accessible to different companies or products.

FIG. 1G shows an example of a system for facilitating genomic or phenotype data exchange, in this case, a system that allows genetic data upload by third-party user(s) that can be selectively accessible to different companies or products.

FIG. 2A shows an example of a system for facilitating genomic or phenotype data exchange, in this case, a system that allows an “entry” company to share genetic data of user with other companies and generate revenue.

FIG. 2B shows an example of a system for facilitating genomic or phenotype data exchange, in this case, a system that allows a user to manage data access to one or more companies.

FIG. 2C shows an example of a system for facilitating genomic or phenotype data exchange, in this case, a system that allows genetic data from multiple companies to be combined so that the combined data can be utilized by another company.

FIG. 2D shows an example of a system for facilitating genomic or phenotype data exchange, in this case, a system that allows genetic data exchange with different data type and/or data format.

FIG. 2E shows an example of a system for facilitating genomic or phenotype data exchange, in this case, a system that is configured to scan genetic data during data exchange so that the information derived from scanning can be utilized by one or more companies.

FIG. 3A illustrates an example of a system that is capable of phenotype data collection with each new product.

FIG. 3B illustrates an example of a system that is capable of displaying health records.

FIG. 3C illustrates an example of a system that is capable of phenotype data collection from a plurality of partners.

FIG. 3D illustrates an example of a system that is capable of phenotype data collection from different consumer and health sources.

FIG. 3E illustrates an example of a system that is capable of delivering value for laboratories by offering a technology and product experience for clients featuring seamless phenotype collection.

FIG. 4 shows a computer system that is programmed or otherwise configured to implement methods provided herein.

FIG. 5 shows an example of unique personalized results being provided for each user.

FIG. 6 shows an example of a user's health data is collected and structured into static health data and dynamic health data.

FIG. 7 shows an example of how the system collects genotype data and/or biomarker data.

FIG. 8 shows an example of how during the data collection process, the system is configured to assign each user with new health attributes (e.g., tags). For example, a user can answer nested questions and receive health attributes (e.g., tags) based on the responses to the questions.

FIG. 9 shows an example of combining a plurality of health attributes to create a health data graph for each user.

FIG. 10 shows an example of labeling datasets used to train machine learning and artificial intelligence models that personalize the information patients receive (e.g., based on genotype data and phenotype data of the individual) in order to help them make better decisions about their health.

FIG. 11 shows an example of personalized action plans tailored to each individual based on the user's health data graph (e.g., static data and dynamic data), such as genotype data, biomarker data, and/or phenotype data.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an” and “the” include plural references unless the context clearly dictates otherwise. For example, the term “a biological sample” includes a plurality of biological samples, including mixtures thereof.

As used herein, the term “subject,” generally refers to an entity or a medium that has testable or detectable genetic information. A subject may be a person or individual. A subject may be a vertebrate, such as, for example, a mammal. Non-limiting examples of mammals include humans, simians, farm animals, sport animals, and pets. A subject may be an organism, such as an animal, a plant, a fungus, an archaea, or a bacteria.

A biological sample may be obtained from a subject. Samples obtained from subjects may comprise a biological sample from a human, animal, plant, fungus, or bacteria. The sample may be obtained from a subject with a disease or disorder, from a subject that is suspected of having the disease or disorder, or from a subject that does not have or is not suspected of having the disease or disorder. The disease or disorder may be an infectious disease, an immune disorder or disease, a cancer, a genetic disease, a degenerative disease, a lifestyle disease, an injury, a rare disease, or an age related disease. The infectious disease may be caused by bacteria, viruses, fungi, and/or parasites. The sample may be taken before and/or after treatment of a subject with a disease or disorder. Samples may be taken during a treatment or a treatment regime. Multiple samples may be taken from a subject to monitor the effects of the treatment over time. The sample may be taken from a subject having or suspected of having a disease or disorder for which a definitive positive or negative diagnosis is not available via clinical tests.

The sample may be obtained from a subject suspected of having a disease or a disorder. The subject may be experiencing unexplained symptoms, such as fatigue, nausea, weight loss, aches and pains, weakness, or memory loss. The subject may have explained symptoms. The subject may be at risk of developing a disease or disorder due to factors such as familial history, age, environmental exposure, lifestyle risk factors, or presence of other known risk factors.

The sample may comprise a biological sample from a human subject, such as stool (feces), blood, cells, tissue (e.g., normal or tumor), urine, saliva, skin swabs, or derivatives or combinations thereof. The biological samples may be stored in a variety of storage conditions before processing, such as different temperatures (e.g., at room temperature, under refrigeration or freezer conditions, at 4° C., at −18° C., −20° C., or at −80° C.) or different preservatives (e.g., alcohol, formaldehyde, potassium dichromate, or EDTA).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic acid (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non nucleotide components. A nucleic acid may be further modified after polymerization, such as by conjugation or binding with a reporter agent.

The nucleic acid molecules may comprise deoxyribonucleic acid (DNA) or ribonucleic acid (RNA) molecules. The DNA or RNA molecules may be extracted from the sample by a variety of methods, such as a FastDNA Kit protocol from MP Biomedicals. The extraction method may extract all DNA molecules from a sample. Alternatively, the extract method may selectively extract a portion of DNA molecules from a sample, e.g., by targeting certain genes in the DNA molecules. Alternatively, extracted RNA molecules from a sample may be converted to DNA molecules by reverse transcription (RT). After obtaining the sample, the sample may be processed to generate a plurality of genomic sequences. Processing the sample may comprise extracting a plurality of nucleic acid (DNA or RNA) molecules from said sample, and sequencing said plurality of nucleic acid (DNA or RNA) molecules to generate a plurality of nucleic acid (DNA or RNA) sequence reads.

The sequencing may be performed by any suitable sequencing method, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, and sequencing-by-hybridization, or RNA-Seq (Illumina). Sequence identification may be performed using a genotyping approach such as an array. As an example, an array may be a microarray (e.g., Affymetrix or Illumina).

The sequencing may comprise nucleic acid amplification (e.g., of DNA or RNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., DNA) to a desired input quantity for subsequent sequencing or genotyping. In some cases, the PCR may be used for global amplification of nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing or genotyping. The PCR may comprise targeted amplification of one or more genomic loci, such as genomic loci corresponding to one or more diseases or disorders such as cancer markers (e.g., BRCA 1 and 2). The sequencing or genotyping may comprise use of simultaneous reverse transcription (RT) and polymerase chain reaction (PCR), such as a OneStep RT-PCR kit protocol provided by Qiagen, NEB, Thermo Fisher Scientific, or Bio-Rad.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase. For example, sequencing or genotyping of DNA molecules may be performed with or without amplification of DNA molecules.

DNA or RNA molecules may be tagged, e.g., with identifiable tags, to allow for multiplexing of a plurality of samples. Any number of DNA or RNA samples may be multiplexed. For example a multiplexed reaction may contain DNA or RNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. For example, a plurality of samples may be tagged with sample barcodes such that each DNA or RNA molecule may be traced back to the sample (and the environment or the subject) from which the DNA or RNA molecule originated. Such tags may be attached to DNA or RNA molecules by ligation or by PCR amplification with primers.

After subjecting the nucleic acid molecules to sequencing, suitable bioinformatics processes may be performed on the sequence reads to generate the plurality of genomic sequences. For example, the sequence reads may be filtered for quality, trimmed to remove low quality, or aligned to one or more reference genomes (e.g., a human genome).

A large number of disorders or diseases may each have their own unique characteristic genetic basis. Thus, analysis of genetic data of human subjects may provide valuable insights into disease cause and risk as well as drug discovery and development in various physiology-related fields. However, launching genetic products can be expensive, complicated, and time-consuming.

The present disclosure is related to genomic or phenotype data access or sharing among multiple users and/or entities. Although analysis of human genetic data may significantly advance our understanding of diseases, there can be concerns about genetic data sharing or disclosure of human subjects. In addition, there may be incomplete oversight of genetic testing or data analysis.

The present disclosure provides systems and methods which may advantageously enable secure, efficient, and convenient sharing of human genomic or phenotype data among multiple users and/or entities. Although analysis of human genetic or phenotype data may significantly advance our understanding of diseases, there can be concerns about sharing of genetic data, phenotype data, or other electronic health record (EHR) data or disclosure of human subjects. In addition, there may be incomplete oversight of genetic testing or data analysis. The systems and methods of the present disclosure can be cloud-based. Such secure, efficient, and convenient sharing of human genomic or phenotype data can advantageously accelerate scientific discovery with high cost efficiencies. For example, healthcare, wellness, and nutrition entities can leverage systems and methods of the present disclosure to provide direct-to-consumer genetic products that add the value of personalization based on users' DNA. The systems and methods of the present disclosure may greatly facilitate removal of barriers such as technology and regulatory, thereby enabling different entities to launch genetic products to end-consumers in a user-friendly way.

In an aspect, the present disclosure provides a computer-implemented method for cloud-based genomic or phenotype data access among a plurality of digital computers comprising a first digital computer of a first user and a second digital computer of a second user, comprising: (a) providing a cloud-based computer system comprising a network interface that is in network communication with the first digital computer of the first user and the second digital computer of the second user; (b) through the network interface, receiving a request from the first digital computer to provide the second user access to a set of genomic or phenotype data, which set of genomic or phenotype data is generated from processing at least one biological sample of a subject; and (c) subsequent to receiving the request in (b), permitting the second user to access at least a subset of the set of genomic or phenotype data through the second computer of the second user.

In some embodiments, operation (c) comprises transferring the at least the subset of the set of genomic or phenotype data to the second computer. In some embodiments, the set of genomic or phenotype data can be stored in the cloud-based computer system, and operation (c) comprises (i) permitting the second user to access the at least the subset of the set of genomic or phenotype data in the cloud-based computer system or (ii) transferring the at least the subset of the set of genomic or phenotype data from the cloud-based computer system to the second computer. In some embodiments, the method further comprises, prior to operation (c), receiving at the cloud-based computer system the set of genomic or phenotype data from the first digital computer. In some embodiments, the method further comprises receiving at the cloud-based computer system a second set of genomic or phenotype data from the second digital computer, which second set of genomic or phenotype data is generated from at least one biological sample of the subject. In some embodiments, the second set of genomic or phenotype data is different than the first set of genomic or phenotype data. In some embodiments, the first user is the subject. In some embodiments, the second user is the subject. The method herein may further comprise receiving an item of value from the second user in exchange for permitting the second user to access the at least the subset of the set of genomic or phenotype data. In some embodiments, the method further comprises providing at least a portion of the item of value to the first user. In some embodiments, the first user may be associated with a first company and the second user may be associated with a second company different from the first company. In some embodiments, the first user may be the subject and the second user may be associated with a company. In some embodiments, operation (b) further comprises using an account of the first user. In some embodiments, the at least the subset of the set of genomic or phenotype data is configured to be used by the second user or a third user to generate health-related information of the subject. In some embodiments, the method further comprises communicating the health-related information of the subject to the first user. In some embodiments, the first user may be the subject or the second user may be the subject. In some embodiments, the method further comprises allowing the first user to manage the set of genomic or phenotype data through the network interface, wherein managing the set of genomic or phenotype data comprises granting access to one or more additional users, reviewing access by the one or more additional users, or manipulating the set of genomic or phenotype data. In some embodiments, the network interface comprises a graphical user interface (GUI). In some embodiments, the network interface is provided via a mobile or web application. In some embodiments, the set of genomic or phenotype data is stored on a private cloud of the first user. In some embodiments, the private cloud comprises a private database structure.

In another aspect, the present disclosure provides a cloud-based method for facilitating genomic or phenotype data exchange, comprising permitting a first entity to access genomic or phenotype data of a second entity over a cloud-based computer system, wherein the genomic or phenotype data is generated from processing at least one biological sample of a subject. In some embodiments, the permission is provided by the second entity. In some embodiments, the permission is provided by the cloud-based computer system. In some embodiments, the cloud-based computer system comprises a network interface. In some embodiments, the set of genomic or phenotype data is configured to be used by the second entity or a third entity to generate health-related information of the subject.

As used herein, a user can be an end-consumer, a company having at least one product that can utilize human genetic data to generate health-related information to an end-consumer, an entity that does not have any product but may also utilize the human genetic data for other purposes such as research, a regulatory agency, a subject from which the biological samples and/or genetic data are obtained; a database where genetic data and phenotype data of subjects are stored, or any other entities that are within the network, thereby in communication with other parts of the system herein.

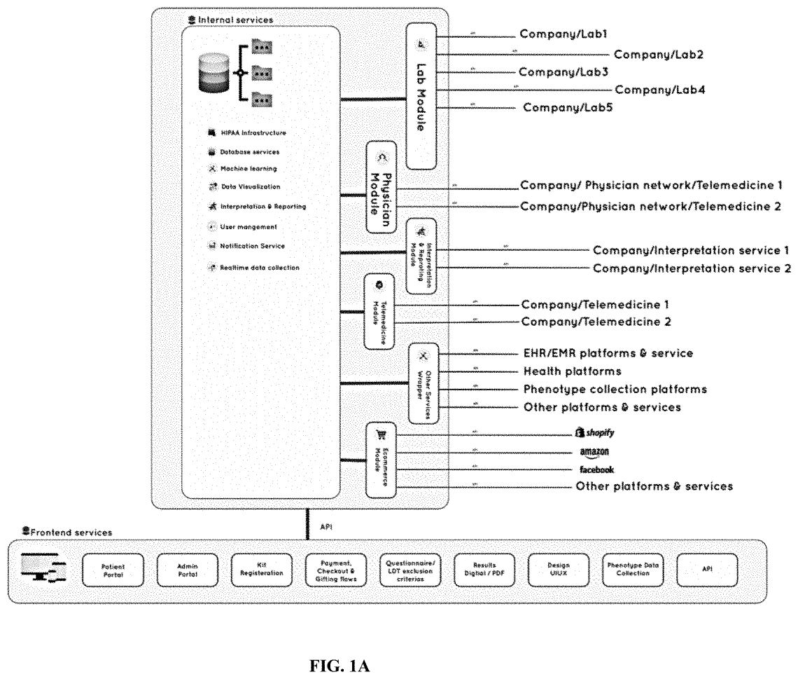

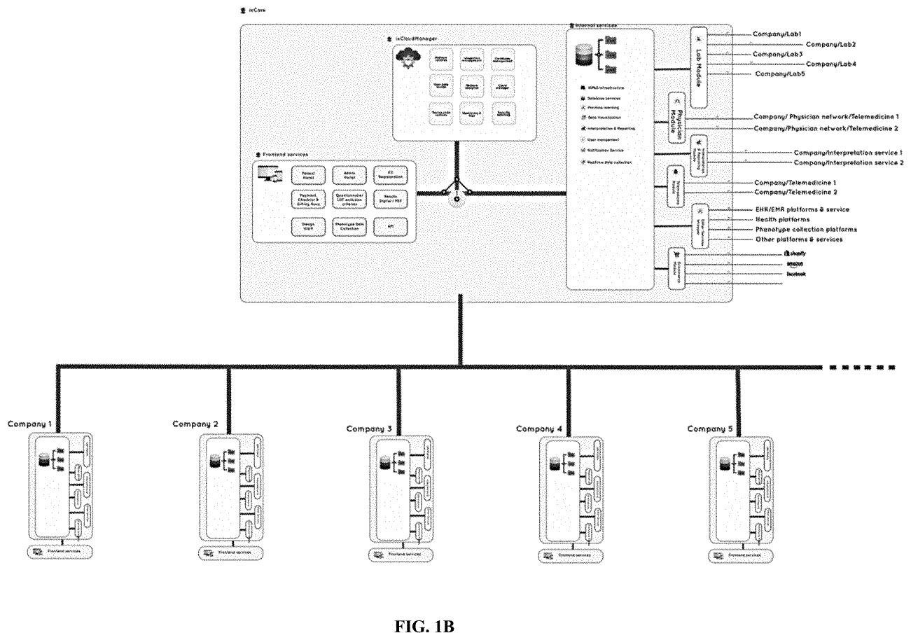

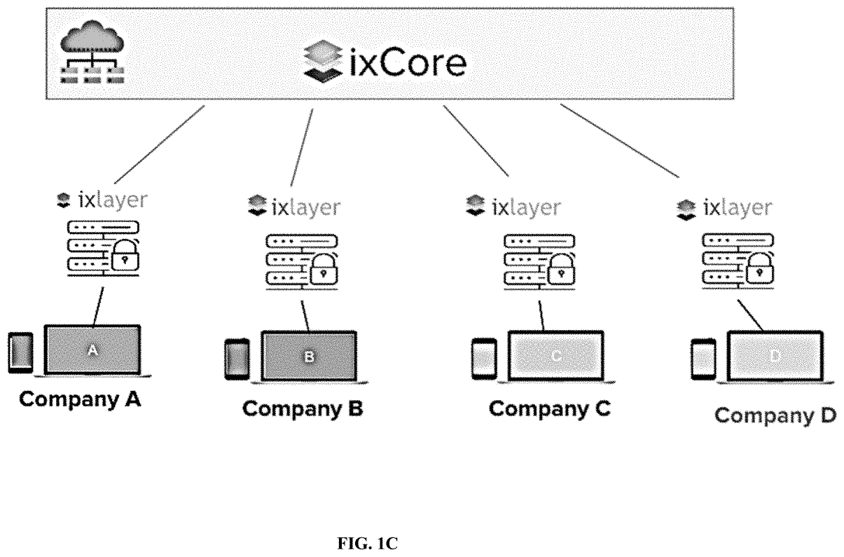

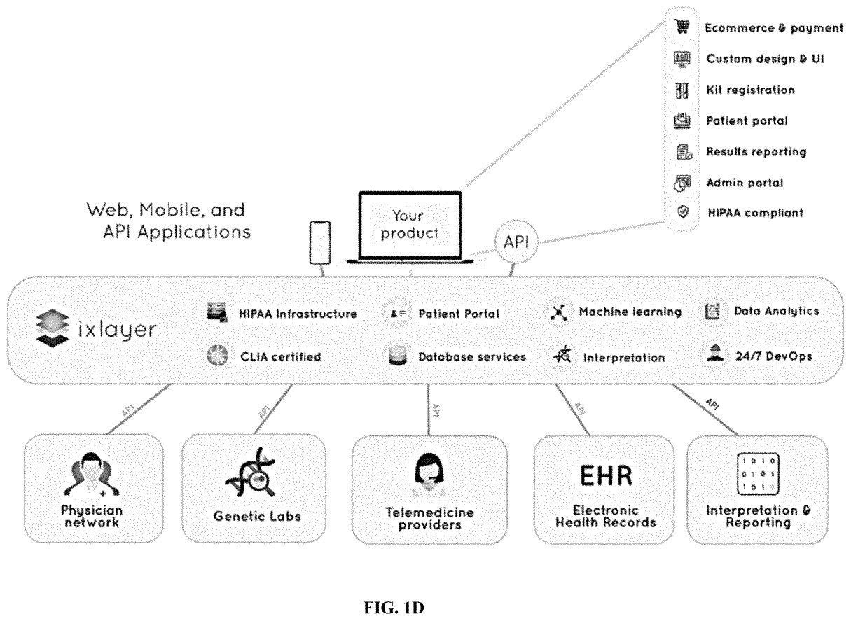

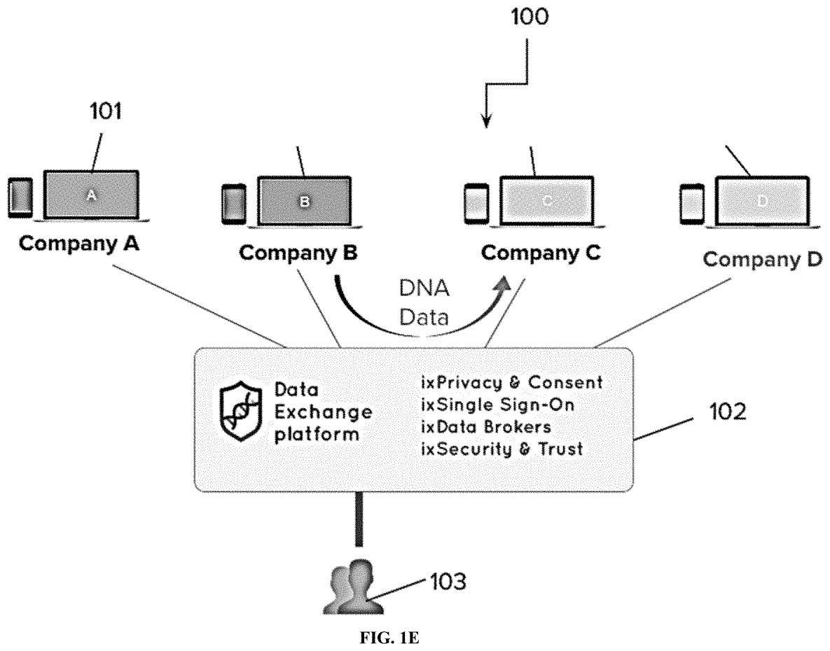

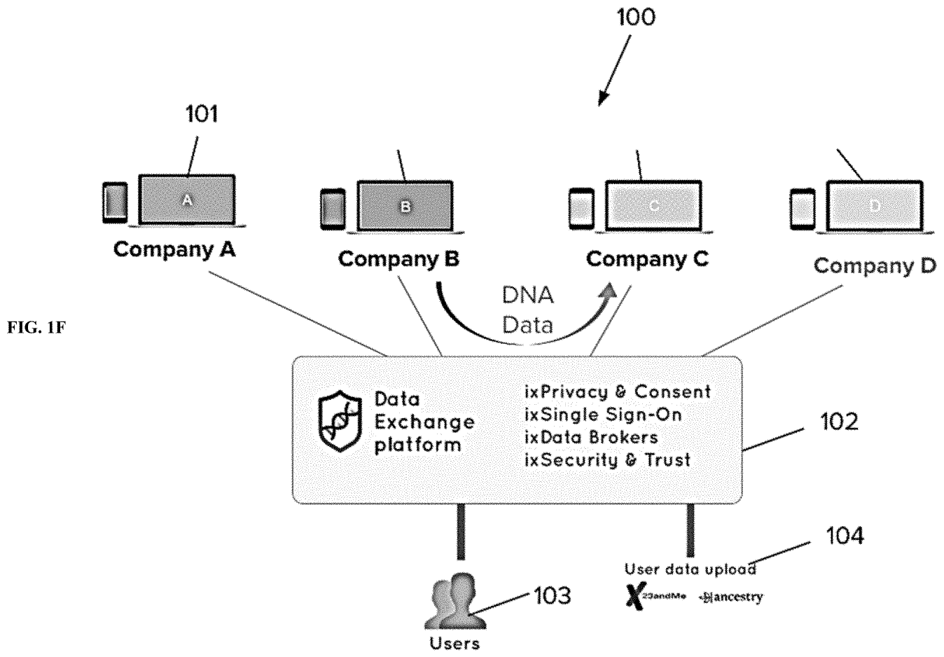

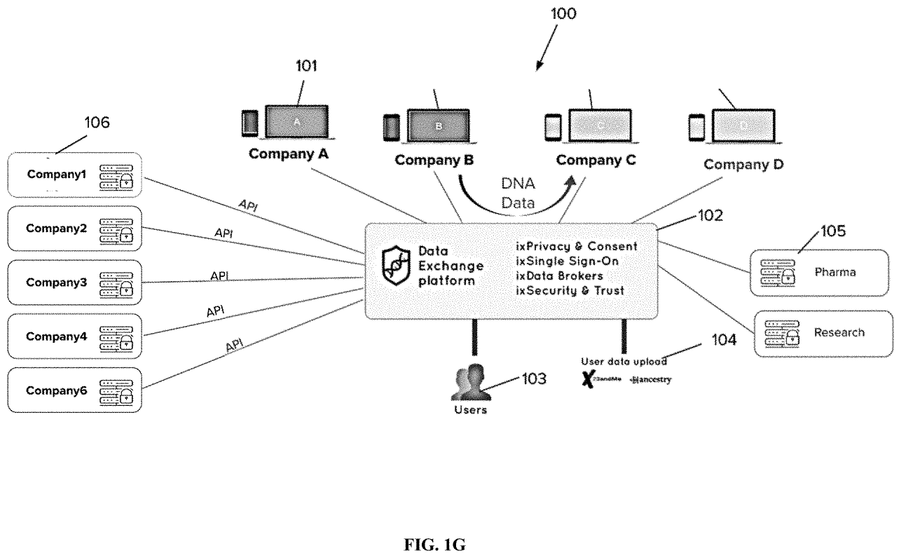

FIG. 1A shows an example of a client virtual private cloud (VPC), which can be implemented using a system 100. Each client can have its own VPC, having its own separate database structure and business logic, such that nothing is shared between two clients. Each VPC can provide internal services including features such as HIPAA (Health Insurance Portability and Accountability Act) infrastructure, database services, machine learning, data visualization, interpretation and reporting, user management, notification service, and real-time data collection, as described herein. Each VPC can provide one or more of such internal services to its client via one or more modules, such as a lab module, a physician module, an interpretation and reporting module, a telemedicine module, a wrapper for other services, and an e-commerce module, as described herein. Each VPC can be provided one or more front-end services via an API, such as a patient portal, an administrator portal, kit registration, payment, checkout, and gifting flows, health questionnaire and exclusion criteria, results in digital or PDF (portable document format) format, design user interface/user experience (UI/UX), phenotype data collection, and API, as described herein. The client VPCs can be integrated with different labs, physician network services, genetic counselor services, interpretation and reporting services, etc.

FIG. 1B shows an example of how a core platform can interface with each of a plurality of VPCs. In this case, five individual VPCs are shown, which correspond to five individual entities or clients. The core platform is in charge of creating (e.g., instantiating) and starting up (e.g., initializing) new environments for future clients and performing health, DevOps, and security monitoring of each of the plurality of client VPCs. In this way, each client VPC is seamlessly encapsulated with separate database structures and business logic, such that nothing is shared between two clients (e.g., for security, privacy, and HIPAA-compliance purposes). The core platform may comprise a cloud manager and/or one or more front-end services. The cloud manager may provide services independently to each client's individual VPC, such as platform updates, integration management, certificate management, user data access, platform analytics, cloud management, source code updates, monitoring and logs, and security patching, as described herein. The one or more front-end services may be provided independently to each client's individual VPC, such as a patient portal, an administrator portal, kit registration, payment, checkout, and gifting flows, health questionnaire and exclusion criteria, results in digital or PDF format, design UI/UX, phenotype data collection, and API, as described herein.

FIG. 1C shows another example of how a core platform can interface with each of a plurality of VPCs. In this case, four individual VPCs are shown, which correspond to four individual entities or clients (Company A, Company B, Company C, and Company D). Each client's individual VPC may receive independent services from the cloud manager, such as platform updates, integration management, certificate management, user data access, platform analytics, cloud management, source code updates, monitoring and logs, and security patching, as described herein. The one or more front-end services may be provided independently to each client's individual VPC, such as a patient portal, an administrator portal, kit registration, payment, checkout, and gifting flows, health questionnaire and exclusion criteria, results in digital or PDF format, design UI/UX, phenotype data collection, and API, as shown in FIG. 1D and described herein.

FIG. 1E shows a system for facilitating genomic or phenotype data exchange 100. The system 100 may function as a hub for all the network nodes 101 (e.g., corresponding to Company A, Company B, Company C, and Company D) and companies to be connected to (e.g., integrated to). The system 100 may comprise a Data Exchange platform 102, which may enable the users 103 (e.g., consumers or patients) to use one account across all companies 101 and products which may provide health-related information based on genetic data analysis. The Data Exchange platform 102 may include a variety of different functionalities, such as single sign-on (SSO), data transfer, data exchange, data brokerage, handling privacy and consent operations, handling security and trust operations, facilitating payments between two companies (e.g., from Company A to Company B) in return for data exchange or data brokerage, scanning data, upload of genetic data and phenotype data, a portal for a user to monitor its data transfers, and integration for third party companies to become part of this network. The system 100 may include one or more client VPCs (e.g., one for each of Company A, Company B, Company C, and Company D). The user may easily and securely transfer genetic data and other data from one company to another (e.g., from company B to company C) and/or from one product to another product. A portal or platform 102 can be provided herein for the user to view patient data and a history of genetic data transfers, and to manage data access by any other users and/or entities. The system may be cloud-based so that at least part of the system includes a cloud. The cloud herein can be a private cloud specific to a user or an entity.

The system 100 herein can be a computer-implemented system for genomic or phenotype data access or exchange among different digital users and/or entities. In such a system, there can be a network interface that is in network communication with digital computers of different users. The network interface may include a portal or a platform as disclosed herein. Through the network interface, a user or entity can receive a request access to a set of genomic or phenotype data from a second user or entity. The set of genomic or phenotype data can be generated from processing at least one biological sample of a subject (e.g., the user). Subsequent to receiving the request, the access can be granted to the user or entity, either by the platform or by the second user or entity who receives the request, to permit the user to access at least a subset of the set of genomic or phenotype data. Granting data access may include transferring at least a subset of the set of genomic or phenotype data to the computer of the second user. The set of genomic or phenotype data can be stored in the cloud-based computer system, and granting data access may include (i) permitting the second user to access the at least the subset of the set of genomic or phenotype data in the cloud-based computer system or (ii) transferring the at least the subset of the set of genomic or phenotype data from the cloud-based computer system to the computer of the second user.

The system 100 herein can provide features such as privacy and consent, single sign-on (SSO), data broker, and security and trust functionalities to the user via the computer network of the system (e.g., Data Exchange). For example, the system can enable the user to use one account (e.g., a single sign-on or SSO) across all companies and products. As another example, the system can enable the user to transfer its data (e.g., genetic and other data) from one company to another (e.g., DNA data from Company B to Company C, as shown). As another example, the system can provide a portal for the user to view history of his data transfers and to revoke access to or delete his data from any company.

As an example, a cloud-based method can be provided to a user for facilitating genomic and phenotype data exchange. The user can use a web-application to log in and directly permit company X to access his genomic or phenotype data over a cloud-based computer system in the application, wherein the genomic or phenotype data is generated from processing at least one biological sample of the user. Alternatively, permission can be provided by the cloud-based computer system which may comprise a network interface. Subsequent to the access being granted, the set of genomic or phenotype data may be configured to be accessed and used by the requesting entity or a third-party entity to generate health-related information of the user. Such information can be delivered to the user so that the user can view and/or manage such information using the same web or mobile application. The Data Exchange platform 102 may include functionality for an authorization and settlement process, which can be similar to a credit card processing approach provided by CyberSource/Visa for developers on an API.

FIG. 1F shows an example of a system in which a user 103 can also upload their own data files 104 via the computer network of the system (e.g., Data Exchange). These data files can contain, for example, their own genetic data, data downloaded from private companies that provide personal genomic or phenotype data (e.g., 23andme or Ancestry) or genomics or phenotype data provided by government, research, or other sources. The data files can be genetic data that is associated with subjects, such as the user, or from data files of a family member or a friend with their consent.

FIG. 1G shows an example of a system 100 in which third party users and/or companies 106 (e.g., companies focusing on analysis of genetic data) can also connect to the portal or platform 102 via the computer network of the system (e.g., Data Exchange). Such connections may be made via an application programmable interface (API). The third party users and/or companies can obtain access to features provided by the portal or platform 102. For example, the third-party user may have an SSO account for accessing all products provided by different companies connected to the platform 102. By the consent of the user, the genetic data of the user or provided by the user can also be shared with other non-genetic organizations 105 such as research institutes or pharmaceutical companies.

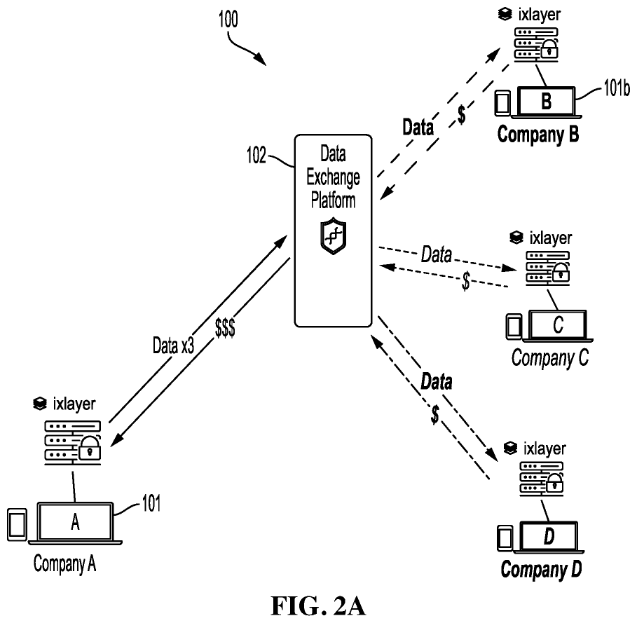

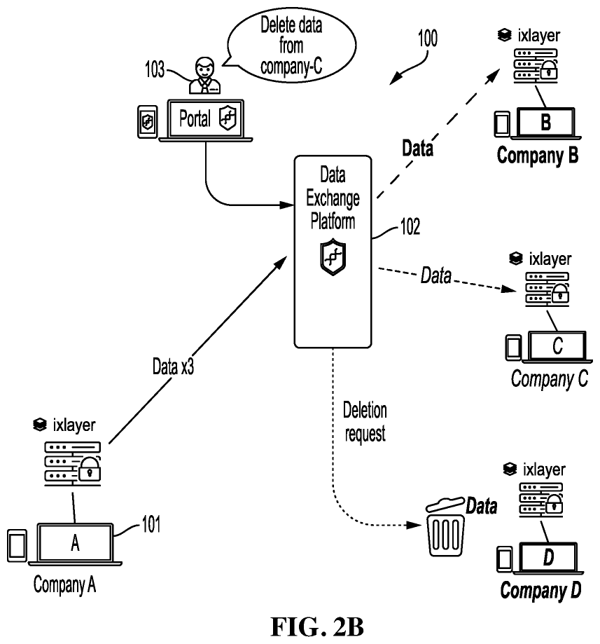

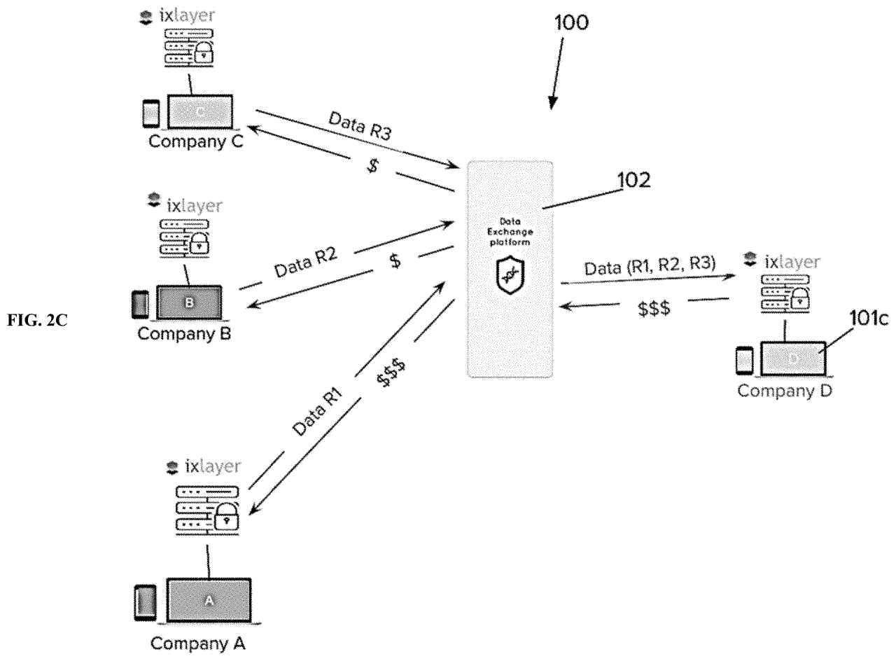

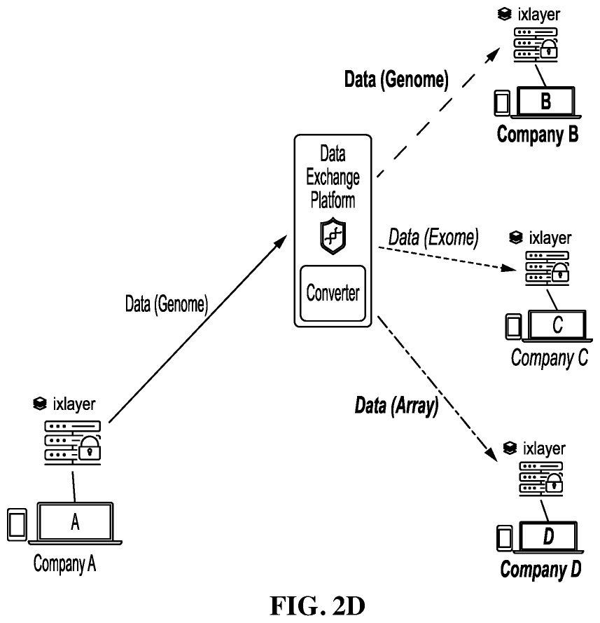

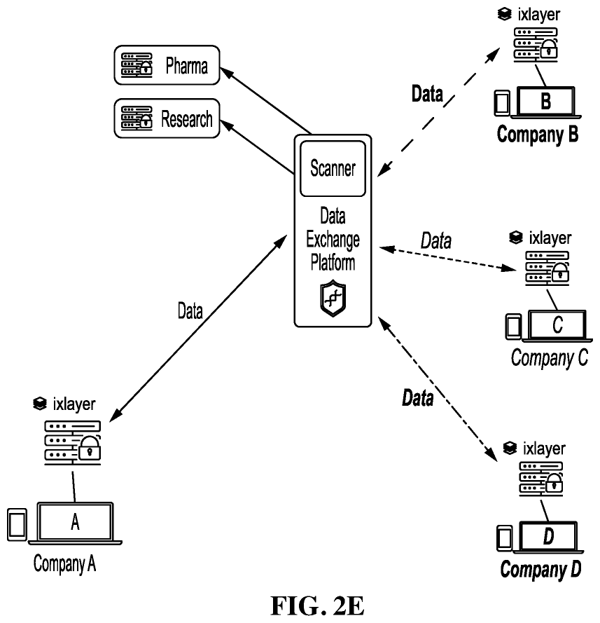

FIG. 2A shows an example of a system 100 disclosed herein for facilitating genomic or phenotype data transfer or exchange, in which, Company A 101 can be the “Entry” company, which means it may be the company that have acquired and analyzed (e.g., sequenced) the genetic data of the user or provided by the user. For example, the products (e.g., genetic tests) provided by Company A 101 can be the first products that the user has purchased. Alternatively, the products provided by Company A 101 can be the first products that have utilized the genetic data associated with the user. After the analyzed (e.g., sequenced) data is ready, the user can, at any point in time, buy any of the other products within the computer network of the system 100 (e.g., the Data Exchange network). As a part of the network, the user may receive a discounted price for products in the system. As part of the second product purchase (e.g., from Company B), the user can consent to the transfer of the genetic data from Company A 101 to Company B 101b. This may allow Company B to instantly interpret at least a portion of the user data and immediately show related test results to the user. At any stage of the process, Company B may compensate (e.g., pay) Company A through the portal or platform 102 for the transfer of the user data with an item of value, for example, an amount of money (e.g., cash or cash equivalents) equal to the portion of the price of the sequencing cost that the user have paid to company A (e.g., about 5%, about 10%, about 20%, about 30%, about 40%, about 50%, about 60%, about 70%, about 80%, or about 90%). Alternatively, the items of value may be coupons, vouchers, credits, IOUs, or other mediums of exchange. The system can automatically perform the payment handling involving such items of value.

Using the systems and methods herein, company A 101 can generate revenue every time the user buys a new product within the network. In this particular case, when the user buys products from company B, company C, and/or company D, company A 101 obtains revenue for transferring the user's genetic data to company B, company C, and/or company D, or for allowing access to the data by company B, company C, and/or company D. This revenue may aggregate to a substantial amount, in some cases exceeding the user acquisition cost and user data analysis (e.g., sequencing or genotyping) cost, which may thereby promote companies to acquire new customers.

The system provided herein may enable users within the network to receive an item of value from the second user in exchange for permitting the second user to access at least a subset of the set of genomic or phenotype data. The method provided herein may further comprise providing at least a portion of the item of value from the second user to the first user or entity. The first user may be associated with a first company, and the second user may be associated with a second company different from the first company. One or both of the first user and the second user may be an end-consumer. For example, the first user may be the subject, and the second user may be associated with a company. The operations herein may further comprise using an account of the user. The at least the subset of the set of genomic or phenotype data may be configured to be used by the second user or a third user to generate health-related information of the subject. The method provided herein may further comprise communicating the health-related information of the subject to the first user. Such communication may be via the portal or platform provided herein. The first user may be the subject, or the second user may be the subject.

FIG. 2B illustrates that using the provided systems and methods for facilitating genetic data exchange, the user 103 can maintain control of the data and can at any point revoke access and request portal data deletion from one or more of the companies with which data has been shared. Upon revoking access or performing the data deletion request from user, the portal or platform 102 can automatically (e.g., via APIs) or manually contact the company 101 and request a deletion. All companies within the network may agree to respect these terms and delete the user data within a reasonable or contractually agreed upon period of time (e.g., 30 days).

The method provided herein may comprise allowing a user to manage the set of genomic or phenotype data through the network interface having a portal or platform, wherein managing the set of genomic or phenotype data may comprise granting access to one or more additional users, reviewing access by the one or more additional users, or manipulating the set of genomic or phenotype data. The network interface may comprise a graphical user interface (GUI). The network interface may be provided via a mobile or web application. The set of genomic or phenotype data can be stored on a private cloud of the first user. The private cloud may comprise a private database structure.

FIG. 2C illustrates that the system and methods for facilitating genetic data exchange can be configured to enable data transfer among multiple sources in the network. The portal or platform 102 can support data transfer from multiple sources, for example, in the case where a portion of the data needed for generating information is missing. In this example, Company D 101c has a test that needs to utilize data from three different genomic regions (e.g., single nucleotide polymorphisms, SNPs) 1, 2, 3, and Company A only sequences region 1, Company B only sequences region 2, and Company C only sequences region 3. The portal or platform 102 can automatically or manually pull the data from all three sources and combine them so the data can be useful for company D (e.g., for analysis or management).

The systems and methods for facilitating genomic or phenotype data transfer can be configured with a data converter to perform data conversion among different data types. FIG. 2D illustrates an example of a system that is capable of data conversion between different data types (e.g., genome data, exome data, and array data) and file formats (e.g., variant call format, VCF), so that different data types can be easily and conveniently transferred among users and/or entities connected to the platform or portal 102.

FIG. 2E illustrates an example of a system in which the platform 102 is configured with a data scanner to scan the genetic or phenotype data as part of the transfer to find user(s) with certain genetic characteristics (e.g., genetic variants) that may be valuable for pharmaceutical companies or research institute. Such users may have particular genetic characteristics (e.g., genetic variants such as single nucleotide polymorphisms (SNPs), insertions or deletions (indels), copy number variation (CNVs), or fusions), phenotypes (e.g., a disease or disorder status), other characteristics found in Electronic Health Record (EHR) data, or a combination thereof. For example, if a given pharmaceutical company is running clinical trials with certain clinical trial enrollment criteria for patients, the data scanner can find and generate a list or database of users that meet the clinical trial enrollment criteria for one or more clinical trials of the pharmaceutical company, based at least in part on an analysis of the individual users' genomic data or phenotype data (e.g., Electronic Health Record (EHR) data). As another example, if a given research institute is seeking patients of certain cohorts to join in research studies, the data scanner can find and generate a list or database of users that meet the cohort criteria for one or more research studies of the research institute, based at least in part on an analysis of the individual users' genomic data or phenotype data (e.g., Electronic Health Record (EHR) data). The data scan can be performed only for users that have consented to be part of clinical trials or research studies. The platform 102 may only scan user data, but not store the user data, as part of the transfer. The platform may support international data transfer, to enable users to transfer their data internationally and to gain access to genetic tests and products that may not be currently available in their market. As an example, a user in China who has been sequenced by BGI may be able to use the system to buy a fertility test that has so far only been available in the United States, or vice versa.

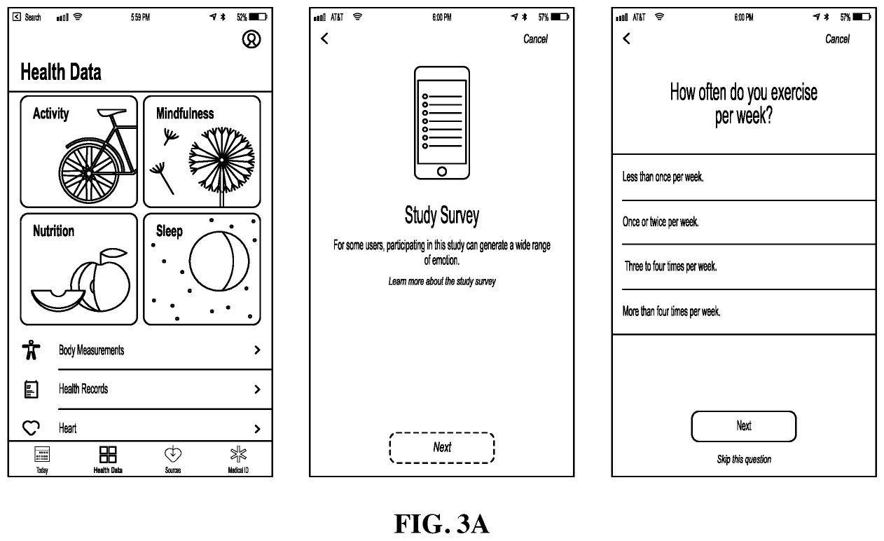

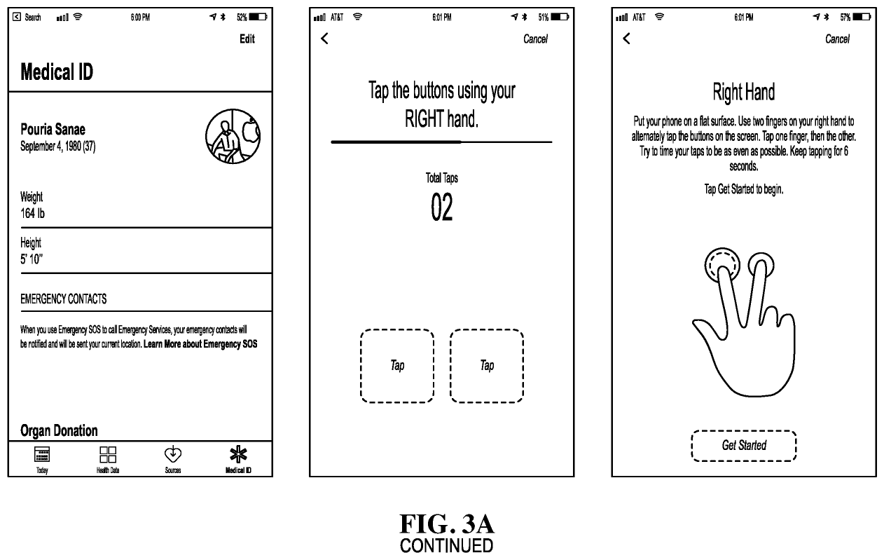

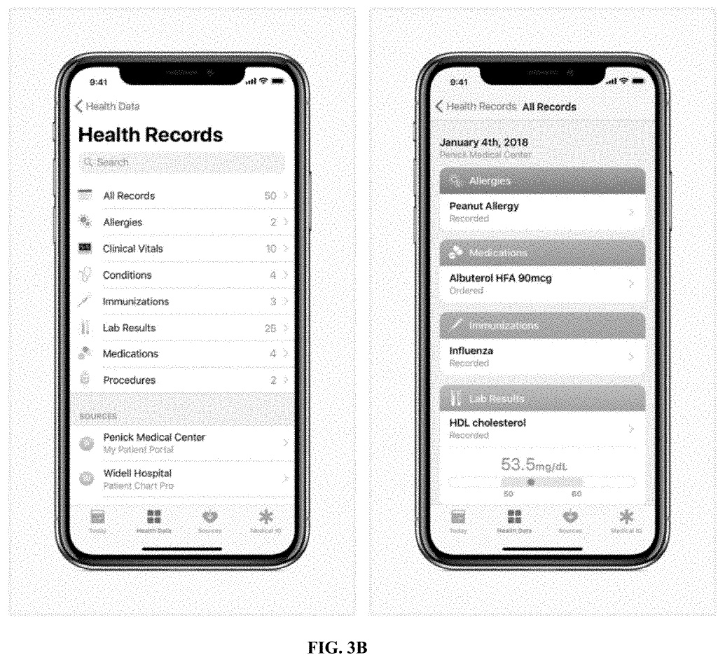

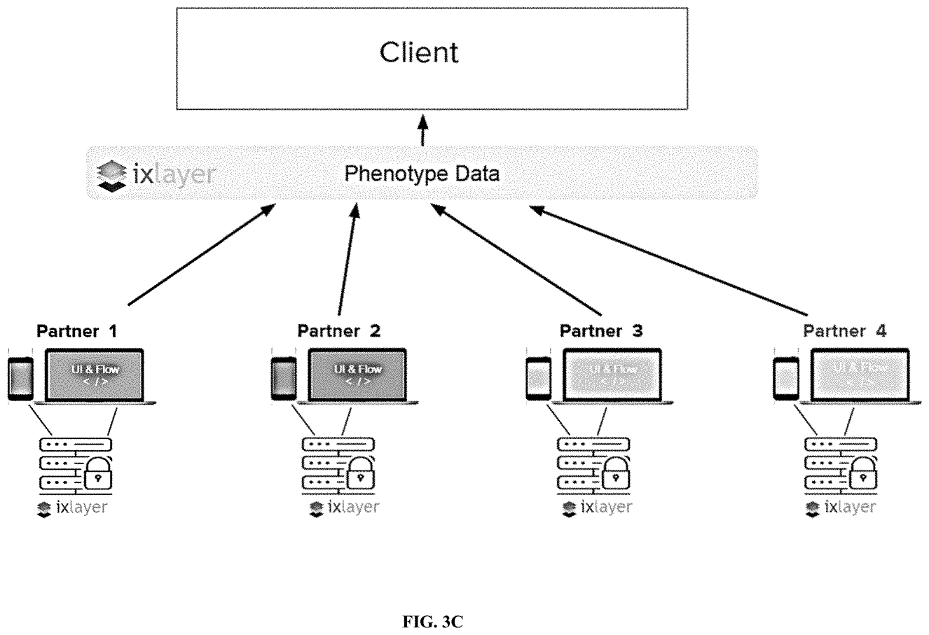

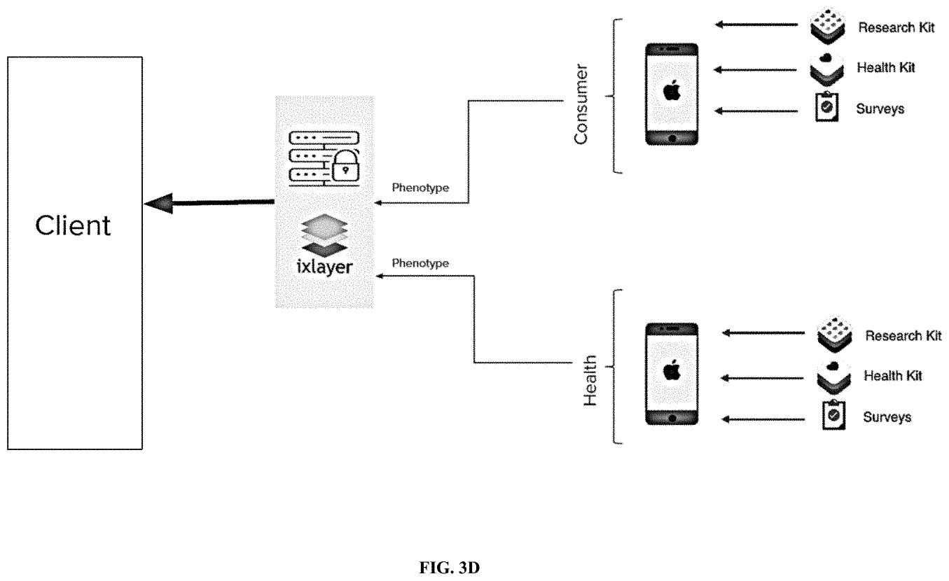

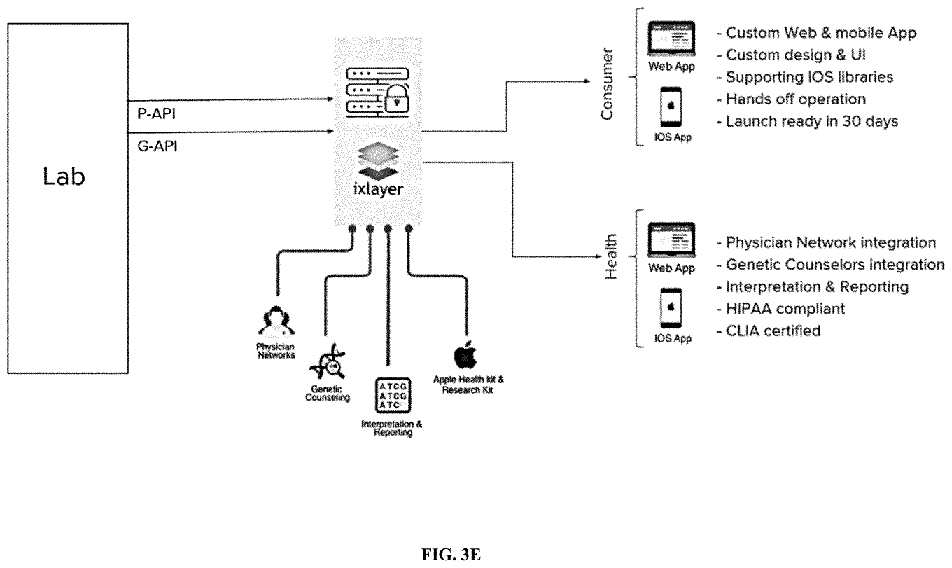

FIG. 3A illustrates an example of a system that is capable of phenotype data collection with each new product. For example, phenotype data collection may include collecting user or patient health data (e.g., including Electronics Health Records (EHR) data) directly from the user or patient. Any phenotype collection device can be integrated with or connected to the system or platform. Such phenotype data collection may be performed in real-time, and may be performed via an API (e.g., a third party centralized application such as Apple HealthKit, Apple ResearchKit, or Apple CareKit) or a mobile application (e.g., Apple iOS or Android) designed to run on a mobile device (e.g., smartphone, tablet computer, smart watch, laptop computer, wearable computer, Apple iPhone, Android phone, Apple iPad, and/or Android tablet). The health data may be related to the activity, mindfulness, nutrition, sleep, body measurements, or other health records of the user or patient. For example, the phenotype data collection may be performed using surveys, in which the user or patient can answer questions presented through the mobile application (e.g., “How often do you exercise per week?”). As another example, the phenotype data collection may be performed by directing the user or patient to enter personal health information into the mobile application (e.g., height, weight, birthdate, blood type, organ donor status, heart rate, blood pressure, cholesterol levels, and/or glucose levels). As another example, the phenotype data collection may be performed by directing the user to interact with the mobile application (e.g., by finger-tapping buttons on the device display such as a smartphone screen). As another example, the phenotype data collection may be performed by the mobile application using one or more sensors such as vital sign sensors (e.g., electrocardiography or ECG sensor, heart rate monitor, blood pressure monitor, pulse oximeter, and/or thermometer) or monitoring or testing devices (e.g., cholesterol monitoring device and/or glucose monitoring device).

FIG. 3B illustrates an example of a system that is capable of displaying health records. Such health records may include collected phenotype data, allergies, clinical vitals, conditions, immunizations, lab results, medications, procedures, and sources of health records.

FIG. 3C illustrates an example of a system that is capable of phenotype data collection from a plurality of partners. In this case, the system can collect or aggregate phenotype data from four different partners (Partner 1, Partner 2, Partner 3, and Partner 4). The collected or aggregated phenotype data can then be transferred or displayed to the client, or otherwise managed or manipulated as desired.

FIG. 3D illustrates an example of a system that is capable of phenotype data collection from different consumer and health sources. In this case, phenotype data can be collected from consumer sources (such as a research kit, a health kit, and surveys) or from health sources (such as a research kit, a health kit, and surveys). The collected or aggregated phenotype data can then be transferred or displayed to the client, or otherwise managed or manipulated as desired.

FIG. 3E illustrates an example of a system that is capable of delivering value for laboratories by offering a technology and product experience for clients featuring seamless phenotype collection. Laboratories can process biological samples from subjects (e.g., users or patients) and obtain genomic data and/or phenotype data, which can be transferred to the platform or system via an API (e.g., P-API or G-API). The platform can facilitate interfacing with physician networks, genetic counselors, interpretation and reporting modules, and health and research kits (e.g., provided by Apple), any or all of which can help process, annotate, or interpret the collected genomic data and/or phenotype data. The patient information (genomic data, phenotype data, and interpretations and reports) can be transferred to consumers (e.g., through a custom web or mobile application that features a custom design and UI, support for iOS libraries, hands-off operation, and rapid launch in about 30 days) or to health care providers (e.g., through a custom web or mobile application that features physician network integration, genetic counselor integration, interpretation and reporting, HIPAA compliance, and CLIA certification).

### Portals or Platforms

The systems and methods provided herein can include a user portal and/or a user platform, as shown in FIGS. 1A-1G, FIGS. 2A-2E, and FIGS. 3A-3E. The portal and/or platform may be part of the network interface. The portal or platform can be used to control connection to the users and/or entities. The portal and/or platform may include a server that includes a digital processing device or a processor that can execute machine code, such as a computer program or algorithm, to enable one or more method steps or operations, as disclosed herein. Such computer programs or algorithms can be run automatically or on-demand based on one or more inputs from the users and/or entities to enable at least partly the genomic or phenotype data exchange.

The portal and/or platform may be used by different entities to launch direct-to-consumer health and wellness products (such as at-home genetic tests), collect real-time user health generated data, recruit new patients and re-engage existing patients, and offer personalized experiences based on users' DNA. For example, such entities may include healthcare, wellness, nutrition, and lifestyle companies that have developed their own genetic laboratory tests. The portal and/or platform may comprise an application program interface (API), and may feature a patient portal (for users to view and manage patient health and genetic or phenotype data), an administrator portal (for administrators to view and manage patient health and genetic or phenotype data), a physician portal (for physicians to view and manage patient health and genetic or phenotype data), a HIPAA infrastructure (e.g., for communication with a physician network), a CLIA-certified infrastructure (e.g., for communication with CLIA-certified genetic labs such as genotyping and sequencing services), machine learning-based database services featuring intelligent reporting (using natural language processing) (e.g., for communication with telemedicine providers, interfacing with electronic health records at clinics, or interpretation and reporting), a health and/or research kit, and a chat bot (e.g., for collection of patient-generated data). The portal and/or platform may offer web application and development libraries, mobile application and development libraries, a custom user interface (UI) designed to fit individual entities' needs, payment handling for web and mobile users, integration with genetic laboratories such as sequencing, genotyping, and diagnostic labs, integration with physician networks, a HIPAA-compliant market place, and ability to launch quickly and easily.

The portal and/or platform may feature full HIPAA compliance, kit registration, a signed-out experience, a sign-in and registration, a patient portal and dashboard, result reporting, a post-result experience, a science information page, a notification service, and an administrator portal with analytics. The portal and/or platform may comprise a module (e.g., a marketplace module) for enabling electronic commerce (e-commerce) features such as integration with e-commerce platforms (e.g., with sales channels on Facebook and Amazon), payment handling and checkout flow, gifting flow, shipping label printing, refund functionalities, and shipping address correction. The portal and/or platform may feature interpretation and result reporting, such as genomic interpretation hosting, results generation, physician approval of results, data visualizations for quick health insights, digital results, and PDF results. The portal and/or platform may feature integration with many different sequencing and genotyping labs. The portal and/or platform may feature functionalities for health products, such as health questionnaires and exclusion criteria, integration with physician networks, integration with GC services, and interaction with HIPAA officers. The portal and/or platform may feature full hands-off operation post launch, such as 24/7 devops, platform updates, integration management, certificate management, source code updates, monitoring and logs, security patching, user data access, platform analytics, post launch bug fixes, and product changes and/or improvements. The portal and/or platform may feature integration with electronic health records (EHR) including genotype and phenotype information (data), for clients that have existing business relationships with the provider, which may feature HL7/FHIR data exchange. The portal and/or platform may feature real-time user or patient data collection, such as integration with wearable devices (e.g., smart watch, Apple Watch, Fitbit, Garman) and health and research kits (e.g., provided by Apple).

The marketplace module may be configured to serve as an e-commerce platform, where all the products and companies that are a part of the Data Exchange network are showcased to users. For example, the marketplace module may be a de-centralized e-commerce platform that offers users the ability to view and purchase different products offered by different companies that are part of the Data Exchange network. Such companies may include, for example, genetic laboratories such as sequencing, genotyping, and diagnostic labs. Further, the marketplace module may select and display to each individual user a customized selection of products offered by the different companies that are part of the Data Exchange network, such that the selected, displayed, and/or recommended products are tailored to offer particular value or relevance to the individual user. For example, such particular relevance to the individual user may be determined based at least in part on an analysis of collected genetic or phenotype data (e.g., Electronic Health Record data) of the individual user (e.g., disease or disorder status), as well as links based on other characteristics, such as ancestry or family relations networks of the individual user, a “like me” network of the individual user, a family history of the individual user, or a same race or ethnicity group of the individual user.

In some embodiments, the marketplace module may provide a mechanism for biopharmaceutical companies to design and conduct clinical trials. For example, such biopharmaceutical companies can use the marketplace as a clinical trial infrastructure that is optimized for rapid trial activation and accrual (e.g., enrollment of new subjects). The clinical trial infrastructure may facilitate aspects of clinical trial enrollment and operations, such as proactive matching and enrollment based on biopharmaceutical partner trials, and analysis and updating of real-time patient lists and databases.

In some embodiments, the marketplace module may provide functionality to personalize employee health for employers. For example, individual users who are employees of a particular employer can use the marketplace to view confidential health insights generated based at least in part on each individual user's genetics or phenotype data, which may include information from genetic counselors and clinical pharmacists. Further, the marketplace module may include tools and services designed to allow individual users to act on the displayed results.

The portal and/or platform may feature the ability for entities to personalize their application experience based on user DNA, thereby enabling entities to better tailor a nutrition plan, workout routine, sleep cycle, or even taste preferences based on their users' genetics. Examples of personalized values based on genetics include weight loss (e.g., BMI, low-fat diets, diabetes risk, saturated fat intake), ancestry (e.g., family history, regional makeup, Neanderthal ancestry), sensitivities (e.g., caffeine metabolism, gluten tolerance, lactose tolerance), fitness (e.g., endurance versus power, hydration levels, muscle composition, injury risk), nutrition (e.g., iron, omega-3 fatty acids, blood glucose, vitamin D), and tastes (e.g., bitter taste, sweet tooth).

The portal and/or platform may feature direct-to-consumer (DTC) products and tests, such as genomic health products and tests (e.g., ACMG 59, fertility, carrier screening, BRCA 1 and 2, cardiovascular, diabetes, Alzheimer's, pharmacogenetics), wellness and nutrition products and tests (e.g., food sensitivity, metabolism, vitamins, inflammation test, sleep and stress, weight loss, wellness panel, glucose), general wellness products and tests (e.g., allergy, heavy metals, cholesterol, heart health, thyroid, drugs and alcohol, diabetes), women's health products and tests (e.g., breast milk DHA, women STIs, ovarian reserve, postmenopause, fertility, prenatal panel), men's health products and tests (e.g., testosterone, men's STIs, testosterone, sexual health, PSA screening, cardio plus).

The portal and/or platform may feature a regulated CLIA and HIPAA compliant technology, such as an end-to-end platform that provides needed regulatory technology to launch a lab-developed product or diagnostic test. The portal and/or platform may feature a patient portal and generated data collection, so that patient-generated health data is collected and reported back in real time from personalized web, mobile platform, and digital devices (e.g., Fitbit, Garmin, etc.). The portal and/or platform may feature genetic counseling on physician approved tests, through integration with physician networks, genomic sequencing and genotyping labs, diagnostic labs, other labs, and telemedicine providers such as genetic counselors. The portal and/or platform may feature hands-off operation including 24/7 DevOps, updates and analytics, user data access, integration versioning, certificate management, and monitoring and logs.

The portal and/or platform may feature an infrastructure solution to be used as a standalone backend solution for web and mobile applications or to be integrated with an existing technology stack of an entity (e.g., a client server). The portal and/or platform may automatically provide or push new updates and improvements to entities or users, such as new features, security patches, operating system updates, updated health kits, updated research kits, updated care kits, API updates, regulatory updates, and CLIA certification updates. The portal and/or platform may feature EHR integration between genotype data and/or phenotype data of a client and a health provider or health system network (e.g., via a data exchange contract). Such EHR integration may use an API of the health provider or health system network to transmit data over a secure virtual private network (VPN), which transmits via HL7 or FHIR.

The portal and/or platform may feature security features, such as a HIPAA compliant BAA (e.g., HIPAA technical safeguards, training for employees, and access to HIPAA compliance officer), operational security (e.g., controlled access through an access policy, two-factor authentication, strong passwords, strictly controlled and monitored network access, use of a bastion host to access servers, logging and auditing and monitoring of network access and server access, performing system updates to patch libraries to prevent penetration attempts), data security (e.g., encrypted communication, databases, and file systems, secure network access through strict firewall rules on VPCs and external, use of encrypted storage of keys with quarterly key rotation), and third party security audits (e.g., quarterly security audits, penetration testing and threat analysis by third party security services).

The portal and/or platform may allow users and/or entities to connect with each other via the portal or platform, such that data exchange can be enabled between any two connected users and/or entities, thereby forming a network of connected users and/or entities. Such data exchange can be secure. The users and/or entities may each have an account for accessing the network and utilizing the functions associated with genomic or phenotype data exchange securely and conveniently.

The portal and/or platform may include a user interface, e.g., graphical user interface (GUI). The portal and/or platform may include a web application or mobile application. The portal and/or platform may include a digital display to display information to the user and/or an input device that can interact with the user to accept input from the user.

### Computer Systems

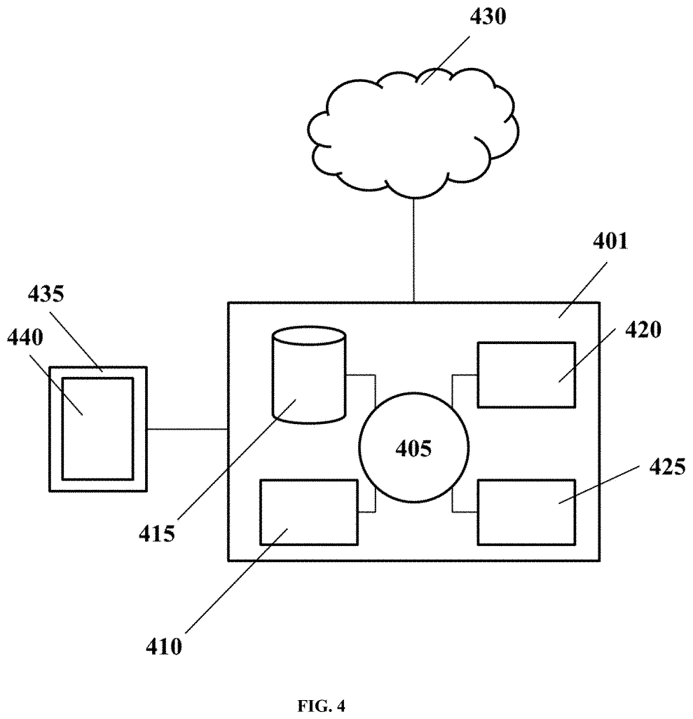

The present disclosure provides computer systems that are programmed to implement methods of the disclosure. FIG. 4 shows a computer system 401 that is programmed or otherwise configured to perform one or more functions or operations for facilitating genomic or phenotype data exchange among different users and/or entities. The computer system 401 can regulate various aspects of the portal and/or platform of the present disclosure, such as, for example, receiving requests from a first digital computer of a first user to provide a second user access to a set of genomic or phenotype data, permitting the user to access at least a subset of the set of genomic or phenotype data through a second computer of the second user, and analyzing genomic or phenotype data or manipulating genomic or phenotype data to generate information (e.g., health-related information) of a subject. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet.

The network 430 in some cases is a telecommunication and/or data network. The network 430 can include one or more computer servers, which can enable distributed computing, such as cloud computing. For example, one or more computer servers may enable cloud computing over the network 430 (“the cloud”) to perform various aspects of analysis, calculation, and generation of the present disclosure, such as, for example, receiving requests from a first digital computer of a first user to provide a second user access to a set of genomic or phenotype data, permitting the user to access at least a subset of the set of genomic or phenotype data through a second computer of the second user, and analyzing genomic or phenotype data or manipulating genomic or phenotype data to generate information (e.g., health-related information) of a subject. Such cloud computing may be provided by cloud computing platforms such as, for example, Amazon Web Services (AWS), Microsoft Azure, Google Cloud Platform, and IBM cloud. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC). The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., a mobile device of the user). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods provided herein can be implemented by way of machine (e.g., computer processor) executable code stored on an electronic storage location of the computer system 401, such as, for example, on the memory 410 or electronic storage unit 415. The machine executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, genomic or phenotype data management. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, receive requests from a first digital computer of a first user to provide a second user access to a set of genomic or phenotype data, permit the user to access at least a subset of the set of genomic or phenotype data through a second computer of the second user, and analyze genomic or phenotype data or manipulate genomic or phenotype data to generate information (e.g., health-related information) of a subject.

### EXAMPLES

### Example 1: A Health Data Graph for Analysis of Static and Dynamic Health Data

Population and precision health generally refers to the outcomes of a group of individuals, including the distribution of such outcomes within the group. Personalized medicine and precision health are among various categories in population health, in which there may be significant value to relating the phenotype (e.g., an observable or measurable trait in an individual) of an individual to the genotype (e.g., map of the individual's genetic information) of the individual. Phenotype may be the result of the genetic code and factors in the environment that impact how an individual develops. Physiological and biochemical properties may also impact how a growing individual matures and develops. The environment in which that an individual grows may greatly affect and changes how the individual matures.

Data may play a vital role in precision health. Initially, the data being analyzed for precision medicine may be obtained from one or a few gene panel tests performed on individuals. Notably, genetics may explain one part of the story, while the other parts may be explained by the phenotype data of an individual.

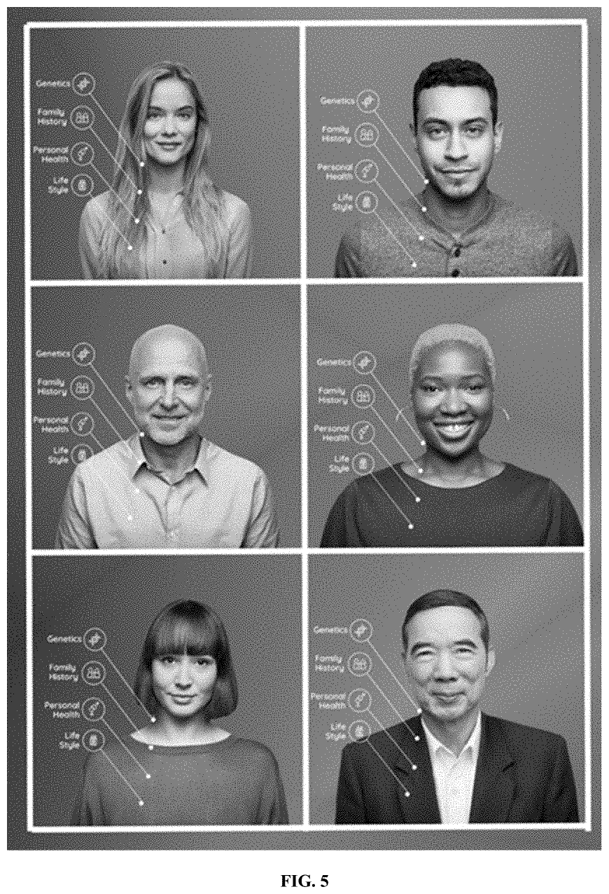

Using systems and methods of the present disclosure, a system (e.g., platform) was designed based on a Health Data Graph structure that depicts relations of each user's static health data (e.g., age, gender, genetics, family history, etc.) and dynamic health data (e.g., life style, daily behavioral choices, medication, etc.). The Health Data Graph structure was used to create a model and representation of each user and the cohort to which the individual belongs. The result is a personalized action plan targeted to each individual. The Health Data Graph platform personalizes the precision health test for each user or participant, and is designed to structure the data taxonomy in an elegant manner to facilitate the collection of health data from a user or users. This enables researchers to conveniently classify data and then provide unique personalized results for each user or users, as shown in FIG. 5.

The system is configured to perform different processes, including identifying and grouping data (e.g., static data and/or dynamic data), collecting data (e.g., static data and/or dynamic data), assigning health attributes (e.g., tags) to a user based on collected data, clustering a user into groups or cohorts and sub-groups or sub-cohorts based on the user's health data graph, and structuring static data and/or ongoing dynamic data for clinical research applications.

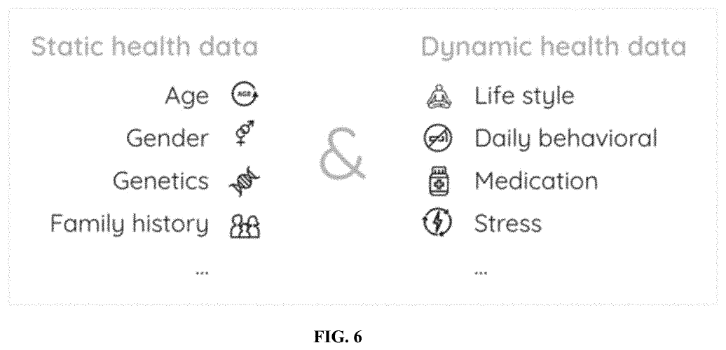

First, the system is configured to identify and group a user's health data (e.g., static data and/or dynamic data). The user's health data is collected and structured into static health data and dynamic health data, as shown in FIG. 6. For example, static health data refers to health data of a user that does not change depending on a user's lifestyle, behavior choices, and other continually changing variables. Static data includes, for example, age, sex, gender, genetics, race, and ethnicity. Dynamic health data refers to continually changing variables including lifestyle data (e.g., daily behavior choices, data obtained from wearable devices (e.g., heart rate, step count, sleep patterns, exercise routines)), electronic health record (EHR) or electronic medical record (EMR) data (e.g., medications, temporary health conditions, ongoing treatments, text notes from doctors, nurses, and other providers), outside factors (e.g., weather and pollution), stress levels, data obtained from social media (e.g., forums, online communities, and mobile applications).

Second, the system is configured to collect data, including static data and dynamic data. The data may include one or more of: genotype or bio-maker (e.g., which can be obtained from a genetic or health test), a health history (e.g., obtained as part of an intake questionnaire), electronic health record data (EHR/EMR), data obtained from wearable technologies and mobile devices, ongoing (engaging) questionnaires and surveys (e.g., obtained through an application dashboard, chatbots, SMS text messages, and e-mails), and data from social media, forums and online communities.

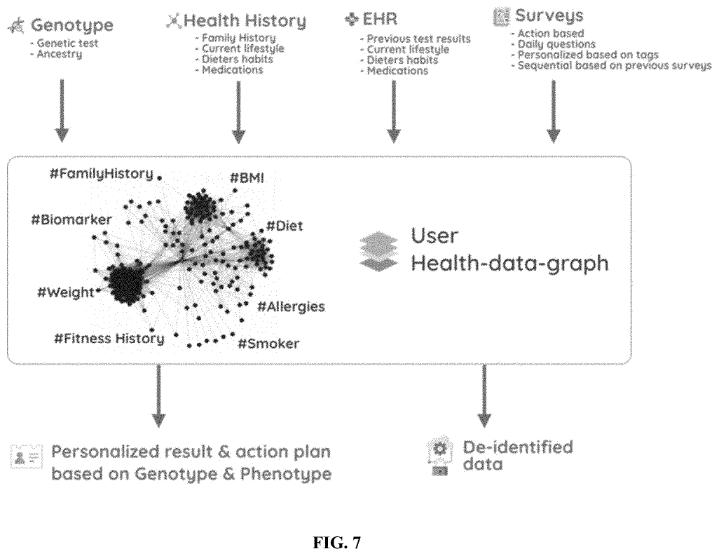

In some embodiments, the system collects genotype data and/or biomarker data (as shown in FIG. 7), which can be obtained from a genetic or health test, a health history of the user as part of an intake questionnaire, electronic health record data, real-time biological data from wearable devices (e.g., Fitbit, Apple health kit, etc.), and social media (e.g., forums and online communities) with the permission of each individual. The data sources utilized in the collection process may include one or more of: genotype data or biomarker data, which can be obtained from a genetic or health test, health history of a user obtained as part of an intake questionnaire, electronic health record data (EHR/EMR), data obtained from wearable devices, and ongoing (engaging) questionnaire or surveys obtained through application dashboard, chatbots, SMS text messages, and e-mails.

Third, the system is configured to assign health attributes (e.g., tags) to a user based on collected data. The health attributes (e.g., tags) may include one or more features such as: using a combination of all health attributes (e.g., tags) to create a health data graph for each user, having a life length (e.g., such that the tag expires after a certain time) for each health attribute, being correlated to each other and either complementing or canceling each other out, becoming smarter over time as more data is collected from an individual, and using the health data graph to create insight for each individual.

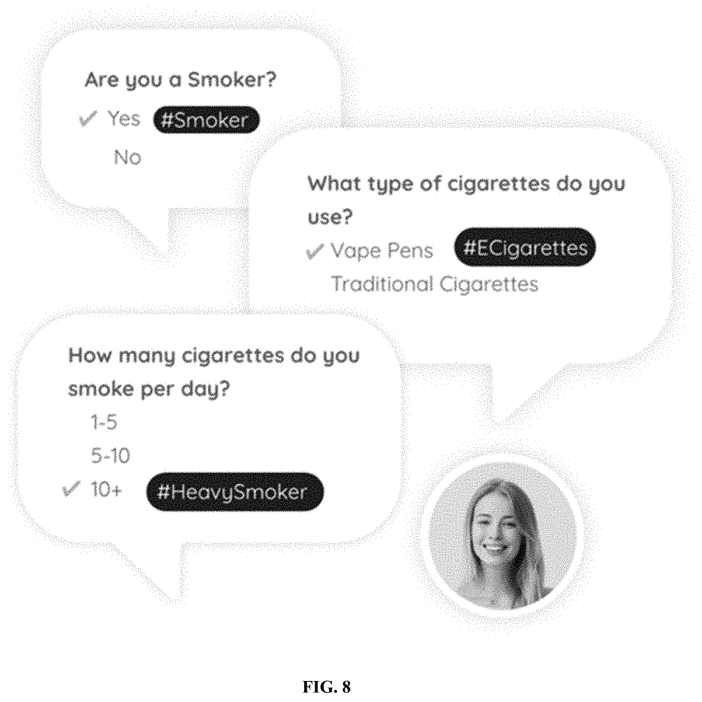

Health attributes (e.g., tags) are assigned to a user based on collected data. During the data collection process, the system is configured to assign each user with new health attributes (e.g., tags). For example, a user can answer nested questions and receive the following health attributes (e.g., tags) based on the responses to the questions: Smoker, Electronic cigarettes, and Heavy smoker (as shown in FIG. 8).

Each health attribute (e.g., tag) has a life length and expiration that can be assigned. After an attribute reaches its life length, the attribute becomes obsolete, and the system either updates the attribute by re-collecting the data (for example, by asking the same questions) or by removing the attribute from the user's health data graph. The attribute can also be updated and replaced at any time. In a real-life example, a user can reduce his or her smoking habit or completely stop smoking at any point in time.

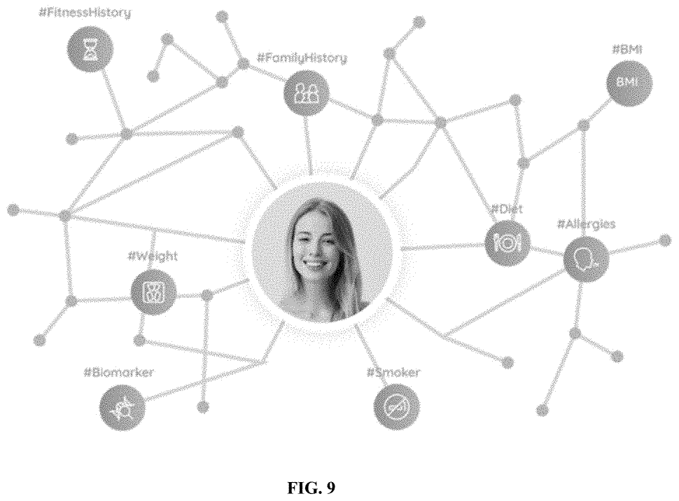

In some embodiments, health attributes (e.g., tags) are correlated to one another. Health attributes can complement or cancel each other out based on their nature. For example, the “#Heavy smoker” attribute is correlates with and complements the related attributes “#Smoker” and “#Electronic cigarettes”. In the case where the user stops smoking, the update of “#Smoker” attribute (from a positive or “yes” value to a negative or “no” value) also cancels out the related “#Heavy smoker” and “#Electronic cigarettes” attributes (e.g., by changing their values from a positive or “yes” value to a negative or “no” value). In some embodiments, a plurality of health attributes can be combined to create a health data graph for each user, as shown in FIG. 9.

Fourth, the system is configured to cluster users into groups or cohorts and sub-groups or sub-cohorts based on the user's health data graph. This clustering may then be used to, for example, provide personalized health results and action plans tailored to each individual based on the user's health data graph (e.g., based on the user's static data and dynamic data) and/or based on the user's genotype data, biomarker data, and/or phenotype data. As another example, this clustering may then be used to, provide personalized dynamic action plans which change with the user's health, lifestyle, and conditions. In some embodiments, the patient-generated data may be harnessed for machine learning-based applications.

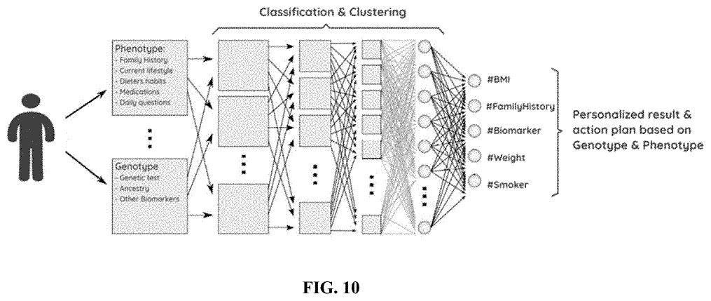

The health data graph enables a creation of a digital representation for each user, and the users can be clustered into different cohorts and sub-cohorts based on each user's health data graph. The system also enables labeling datasets used to train machine learning and artificial intelligence models that personalize the information patients receive (e.g., based on genotype data and phenotype data of the individual) in order to help them make better decisions about their health, as shown in FIG. 10. Further, the system provides a mechanism to collect additional data for a specific cohort of users. As an example, a questionnaire or survey may be pushed to all users that have certain characteristics, such as one or more of: certain genetic variations (e.g., as indicated via genetic testing results), high cholesterol as indicated in the user's blood test, a high heart rate as indicated by data collected from wearable devices, a smoker status based on previous surveys, and being prescribed or taking certain drugs or medications.

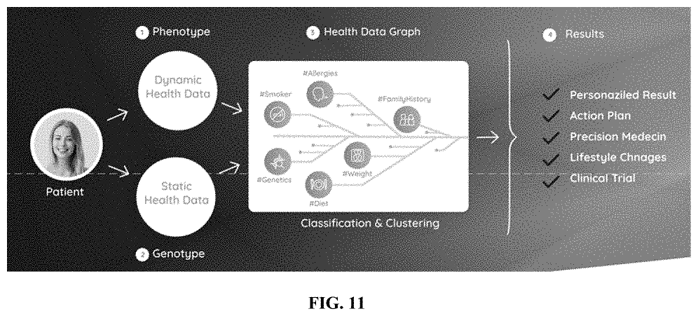

In some embodiments, the system also enables personalized action plans tailored to each individual based on the user's health data graph (e.g., static data and dynamic data), such as genotype data, biomarker data, and/or phenotype data, as shown in FIG. 11. These action plans are dynamic by nature and can change as the user's health, lifestyle, and conditions change (e.g., improve or worsen). Examples of such change include: a user that is gradually getting off a medication as the user's health improves, a pre-diabetic patient who is provided certain nutrition instructions based on his or her health data graph, a set of daily routines and nutrition guidelines and instructions for patients with gestational diabetes, based on their microbiome and other parameters of their health-data graph, and improving outcomes for patients with chronic disease by enabling them to intelligently manage their daily routine in between office visits and health tests.

Fifth, the system is configured to structure static data and ongoing dynamic data for clinical research applications. The system allows researchers to easily query, manipulate, and search the data in a fully aggregated and de-identified manner to ensure that the privacy of each participant is protected.

Using systems and methods of the present disclosure, the dynamic nature of users' clinical data is captured, and trend monitoring of a cohort is performed based on changes in one or more of their attributes, such as a medication change, a reduction of stress, an environmental change, a change in routine nutrition, and a behavioral change.

The system enables researchers to mix and match the different options to investigate the effects of a genotype on multiple traits, to investigate multiple genotypes that affect the same trait, or to evaluate the effect of individual microbiome. This can be achieved by selecting genotype data, phenotype data, or combinations thereof, and plotting heat maps that comprise a visual representation of the data, thereby facilitating a comparative analysis of interaction effects of various genotypes and phenotypes.

In summary, using systems and methods of the present disclosure, personalized results and action plans are generated for end users. In the above example, a user who is a heavy smoker and uses electronic cigarettes has his or her information combined with genotyping information (e.g., a set of genetic variants), and an action plan that is targeted towards offering customized preventive care is provided to the specific individual. Further, by using the health data graph, the system is able to generate and analyze rich clustered datasets for population health studies. The availability of health data graph clusters enables ongoing research and collection of phenotypic data on a regular or continuous basis from each user or participant. Therefore, it is possible to collect new data through the feedback loop, as new phenotypic questions can be asked of each user, based on the genetic biomarkers as well as previous responses provided through the platform. Further, the health data graph enables organizations to de-identify the entire data sets (both static data and dynamic data), thereby enabling structured data to be conveniently exported different machine learning or other scientific tools to be used to perform further research studies.

### Example 2: Using a Health Data Graph for Analysis of a User's Static and Dynamic Health Data

Using systems and methods of the present disclosure, a health data graph system is used for analysis of a user's health data. This analysis procedure comprises onboarding the user, data structuring, risk assessment and test recommendation, testing the user, generating a clinical report for the user, generating personalized and dynamic action plans for the user, performing ongoing data collection and generating dynamic action plans, and training artificial intelligence and machine learning algorithm with improved dynamic models.

First, the user is onboarded to a program. This program may include one or more of: a pre-testing phase of a precision health test, a research study for a population health program, a companion diagnostic testing for the safe and effective use of a corresponding drug or biological product within personalized medicine, or another type of health test. Next, the collection process for that specific patient starts. The data collection may be from one or more of the following sources: health history and family history (e.g., obtained as part of an intake questionnaire), health data of relatives, electronic health record data (EHR/EMR), historical data obtained from wearable devices, a chatbot interaction for data collection, and other data sources.

Second, data structuring is performed by dividing the collected data into static data and dynamic data, as described above. The system runs through both data sets and generates health attributes (e.g., tags) for the patient from both data sets. Attributes can have a relation to one another, and health attributes generated from dynamic data sets may have a pre-determined limited duration of time to be actionable. Examples of health attributes include: [Attribute name: #Smoker or #Non-smoker], [Relation to secondary attributes: #ECigarettes, #HeavySmoker. #Nicotine, etc.], [Attribute life length: #1 week, #2 weeks, #4 weeks, #2 months, #3 months, #4 months, #5 months, #6 months, #7 months, #8 months, #9 months, #10 months, #11 months, #1 year], [Attribute source: #HealthQA], [Attribute model: #Simple], and [Attribute weight: 100 lb, 120 lb, 140 lb, 160 lb, 180 lb, 200 lb, 220 lb, 240 lb, etc.]. A set of these attributes for a particular user are combined to generate a health data graph for a user.

Third, risk assessment and test recommendation are performed for a user. The health data graph (e.g., a set of attributes for a single individual or patient) are fed into artificial intelligence and machine learning algorithms, to evaluate key risk factors such as personal and family history and to match individuals to personalized genetic (or non-genetic) testing recommendations. The system matches an individual's unique health data graph profile to the appropriate genetic tests for him or her. As a result, insights on which genetic tests (or other health tests) are valuable for the individual are provided, thereby enabling more informed decisions and planning.

Fourth, the user is tested. A health provider or clinical staff orders the test for a patient based on the outcome of the test recommendation. This may also be initiated by the patient. The sample collection process can be performed at home or in a clinical setting. As a result of the testing, genotype data or biomarker data (e.g., from a genetic or health test) and a lab result for the test are obtained.

Fifth, a clinical report and action plan are generated. Data structuring is performed on the patient's test results, and the structured data is added as static data (e.g., for genetic reports) and/or as dynamic data (e.g., for blood or microbiome data) to the health attributes. The combination of test results and the health data graph enables a clinical report to be generated based on the user's phenotype data, biomarker data, and/or genotype data.

Sixth, personalized and dynamic action plans are tailored to each individual based on his or her health data graph (static data and dynamic data), such as genotype data, biomarker data, and phenotype data. For example, the action plan for a first user having a set of certain genetic variants, high cholesterol indicated by his or her blood test, a high heart rate based on data collected from wearable devices, a smoker status based on previous surveys, and who is on a certain drug or medication, is very different from a second user with the same genetic variants, high cholesterol indicated by his or her blood test, a high heart rate based on data collected from wearable devices, a non-smoker status based on previous surveys, and who is not on a drug treatment.

A classification and clustering engine processes the structured data using AI and machine learning algorithms to generate an action plan that matches the user to point, as shown in FIGS. 10-11.

Sixth, ongoing data collection is performed, and dynamic action plans are generated. The data collection process is an ongoing process for all the users in the program; therefore, the health data graph of a user is constantly changing based on the user's dynamic data. The process is also constantly updating, and the generated personalized action plans are also dynamic in nature.

These action plans can change as the user's health, lifestyle, and conditions improve or worsen. Examples of such change include: a user that is gradually getting off a medication as the user's health improves, a pre-diabetic patient who is provided certain nutrition instructions based on his or her health data graph, a set of daily routines and nutrition guidelines and instructions for patients with gestational diabetes, based on their microbiome and other parameters of their health-data graph, and improving outcomes for patients with chronic disease by enabling them to intelligently manage their daily routine in between office visits and health tests.

Seventh, AI and machine learning algorithms are trained with improved dynamic models. The health data graph is a digital representation for each user, and users are clustered in different cohorts and sub-cohorts. These datasets are used to train machine learning and artificial intelligence models that personalize the information patients receive in order to help them make better decisions about their health. Therefore, value is created in the form of models that can be based not just on one data set, but on a duration of time. Examples include how a patient with certain genetic variants experience the effect by a drug in a short-term and long-term study. The health data graph can also be combined with the raw genetic data of users to unlock new discoveries based on clustering users and patients into cohorts and finding correlations between their genotyping data, biomarker data, and/or phenotype data. Based on these correlations, discoveries, and other analyses, therapy recommendations are generated for individual users.

### Example 3: An End-to-End Workflow of the Health Data Graph

Health organization adopt health-data graph

Patient visits health provider

Health provider recommends certain treatment action based on patients health history and genomic test results

Patient goes back home and may or may not adopt the treatment plan

Surveys are sent to patient to answer certain health questions via email/txt

Biometric data is collected from patient via wearable devices/social media/etc

Health-Data Graph is modified based on 5 and 6 and patient may be moved to different cohort

The treatment plan may be completely different on the patient's next visit to health provider

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A computer-implemented method for cloud-based genomic or phenotype data access among a plurality of digital computers comprising a first digital computer of a first user and a second digital computer of a second user, comprising:
(a) providing a cloud-based computer system comprising a network interface that is in network communication with said first digital computer of said first user and said second digital computer of said second user;
(b) through said network interface, receiving a request from said first digital computer to provide said second user access to a set of genomic or phenotype data, which set of genomic or phenotype data is generated from processing at least one biological sample of a subject; and
(c) subsequent to receiving said request in (b), permitting said second user to access at least a subset of said set of genomic or phenotype data through said second computer of said second user.

2. The method of claim 1, wherein (c) comprises transferring said at least said subset of said set of genomic or phenotype data to said second computer.

3. The method of claim 1, wherein said set of genomic or phenotype data is stored in said cloud-based computer system, and wherein (c) comprises (i) permitting said second user to access said at least said subset of said set of genomic or phenotype data in said cloud-based computer system or (ii) transferring said at least said subset of said set of genomic or phenotype data from said cloud-based computer system to said second computer.

4. The method of claim 1, further comprising, prior to (c), receiving at said cloud-based computer system said set of genomic or phenotype data from said first digital computer.

5. The method of claim 4, further comprising receiving at said cloud-based computer system a second set of genomic or phenotype data from said second digital computer, which second set of genomic or phenotype data is generated from at least one biological sample of said subject.

6. The method of claim 5, wherein said second set of genomic or phenotype data is different than said first set of genomic or phenotype data.

7. The method of claim 1, wherein said first user is said subject or said second user is said subject.

8. (canceled)

9. The method of claim 1, further comprising receiving an item of value from said second user in exchange for permitting said second user to access said at least said subset of said set of genomic or phenotype data.

10. The method of claim 9, further comprising providing at least a portion of said item of value to said first user.

11. The method of claim 1, wherein said first user is associated with a first company and said second user is associated with a second company different than said first company.

12. The method of claim 1, wherein said first user is said subject and said second user is associated with a company.

13. The method of claim 1, wherein (b) further comprises using an account of said first user.

14. The method of claim 1, wherein said at least said subset of said set of genomic or phenotype data is configured to be used by the second user or a third user to generate health-related information of said subject.

15. The method of claim 14, further comprising communicating said health-related information of said subject to said first user.

16. (canceled)

17. The method of claim 1, further comprising allowing said first user to manage said set of genomic or phenotype data through said network interface, wherein managing said set of genomic or phenotype data comprises granting access to one or more additional users, reviewing access by said one or more additional users, or manipulating said set of genomic or phenotype data.

18. The method of claim 1, wherein said network interface comprises a graphical user interface (GUI).

19. The method of claim 1, wherein said network interface is provided via a mobile or web application.

20. The method of claim 1, wherein said set of genomic or phenotype data is stored on a private cloud of said first user.

21.-26. (canceled)

27. A computer system for cloud-based genomic or phenotype data access among a plurality of digital computers comprising a first digital computer of a first user and a second digital computer of a second user, comprising:
a cloud-based computer system comprising a network interface that is in network communication with said first digital computer of said first user and said second digital computer of said second user; and
one or more computer processors operatively coupled to said cloud-based computer system, wherein said one or more computer processors are individually collectively programmed to:
(i) through said network interface, receive a request from said first digital computer to provide said second user access to a set of genomic or phenotype data, which set of genomic or phenotype data is generated from processing at least one biological sample of a subject; and
(ii) subsequent to receiving said request, permit said second user to access at least a subset of said set of genomic or phenotype data through said second computer of said second user.

28.-52. (canceled)

53. A non-transitory computer-readable medium comprising machine-executable code that, upon execution by one or more computer processors, implements a method for cloud-based genomic or phenotype data access among a plurality of digital computers comprising a first digital computer of a first user and a second digital computer of a second user, said method comprising:
(a) providing a cloud-based computer system comprising a network interface that is in network communication with said first digital computer of said first user and said second digital computer of said second user;
(b) through said network interface, receiving a request from said first digital computer to provide said second user access to a set of genomic or phenotype data, which set of genomic or phenotype data is generated from processing at least one biological sample of a subject; and
(c) subsequent to receiving said request in (b), permitting said second user to access at least a subset of said set of genomic or phenotype data through said second computer of said second user.

54.-73. (canceled)
